(19) United States
(12) Patent Application Publication
(10) Pub. No.: US 2008/0153822 A1
(43) Pub. Date: Jun. 26, 2008

(54) Methods of treating pain

(76) Inventors: Martin Augustin, Seefeld-Hechendorf (DE); Gabriele Stumm, Unterhaching (DE); Hans Allgeier, Loerrach-Haagen (DE); Andreas Marquardt, Riemen (DE); Lutz Zeitlmann, Munchen (DE)

Correspondence Address: Olson & Cepuritis, LTD., 20 NORTH WACKER DRIVE, 36TH FLOOR, CHICAGO, IL, 60606, US

(21) Appl. No.: 11/998,221

(22) Filed: Nov. 29, 2007

Related U.S. Application Data
(60) Provisional application No. 60/872,128, filed on Nov. 30, 2006.

(51) Int. Cl. A61K 31/5377; A61P 43/00; A61K 31/496; A61K 31/505

(52) U.S. Cl. 514/235.8; 514/275; 514/252.14

(57) ABSTRACT

The present invention provides compositions and methods for treating pain using a compound of general Formula I:

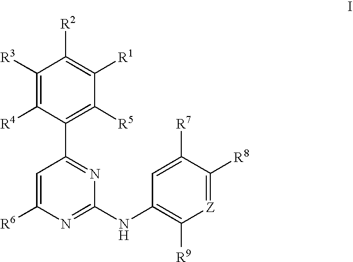

wherein the substituents thereof are as described herein.

### CROSS-REFERENCE TO RELATED APPLICATIONS

This application claims the benefit of U.S. Provisional Application Ser. No. 60/872,128, filed on Nov. 30, 2006, which is incorporated by reference.

### FIELD OF THE INVENTION

The present invention relates to inhibitors of cyclin-dependent kinases and therapeutic applications thereof. Furthermore, the invention relates to methods of preventing and/or treating any type of pain comprising the administration of an effective amount of at least one inhibitor of cyclin-dependent kinases.

### BACKGROUND OF THE INVENTION

Cyclin-dependent protein kinases (“CDKs”) constitute a family of well-conserved enzymes that play multiple roles within the cell, such as cell cycle regulation and transcriptional control (Science 1996, Vol. 274:1643-1677; Ann. Rev. Cell Dev. Biol., 1997, 13:261-291.

Some members of the family, such as CDK1, 2, 3, 4, and 6 regulate the transition between different phases of the cell cycle, such as the progression from a quiescent stage in G1 (the gap between mitosis and the onset of DNA replication for a new round of cell division) to S (the period of active DNA synthesis), or the progression from G2 to M phase, in which active mitosis and cell division occur. Other members of this family of proteins, including CDK7, 8, and 9 regulate key points in the transcription cycle, whereas CDK5 plays a role in neuronal and secretory cell function.

CDK complexes are formed through association of a regulatory cyclin subunit (e.g., cyclin A, B1, B2, D1, D2, D3, and E) and a catalytic kinase subunit (e.g., cdc2 (CDK1), CDK2, CDK4, CDK5, and CDK6). As the name implies, the CDKs display an absolute dependence on the cyclin subunit in order to phosphorylate their target substrates, and different kinase/cyclin pairs function to regulate progression through specific portions of the cell cycle.

CDK9 in association with its cyclin partners (cyclin T1, T2a, T2b, or K) constitutes the catalytic component of the positive P-TEFb protein kinase complex that functions during the elongation phase of transcription by phosphorylating the carboxyl-terminal domain (CTD) of the largest subunit of RNA polymerase II. P-TEFb acts in concert with positive transcription factor NfκB as well as negative transcription factors, thus overcoming a block of transcriptional elongation (Liu and Herrmann 2005).

It is known that cell-cycle dysregulation, which is one of the cardinal characteristics of neoplastic cells, is closely associated with genetic alteration and deregulation of CDKs and their regulators, suggesting that inhibitors of CDKs may be useful as therapeutics for proliferative diseases, such as cancer. Thus, small molecule inhibitors targeting CDKs have been the focus of extensive interest in cancer therapy (Current Opinion in Pharmacology, 2003(3): 362-370). The ability of inhibiting cell cycle progression suggests a general role for small molecule inhibitors of CDKs as therapeutics for proliferative diseases, such as cancer. While inhibition of cell cycle-related CDKs is clearly relevant in oncology applications, this may not be the case for the inhibition of RNA polymerase-regulating CDKs. Recently, inhibition of CDK9/cyclin T function was linked to prevention of HIV replication and the discovery of new CDK biology thus continues to open up new therapeutic indications for CDK inhibitors (Sausville, E. A. Trends Molec. Med. 2002, 8, S32-S37) such as, for example, the treatment of viral infections (WO 02/100401). CDK inhibitors could conceivably also be used to treat other conditions such as immunological diseases and neurodegenerative diseases, amongst others.

More than 50 pharmacological CDK inhibitors have been described, some of which have potent antitumor activity (Current Opinion in Pharmacology, 2003(3): 362-370). A comprehensive review about the known CDK inhibitors may be found in Angew. Chem. Int. Ed. Engl. 2003, 42(19):2122-2138.

Indolinone derivatives and induribin derivatives, which are useful as cyclin-dependent kinase inhibitors, have been disclosed in WO 02/081445 and WO 02/074742. Additionally, CDK inhibitors for various therapeutic applications have been described in WO2005/026129.

Known CDK inhibitors may be classified according to their ability to inhibit CDKs in general or according to their selectivity for a specific CDK. Flavopiridol, for example, acts as a “pan” CDK antagonist and is not particularly selective for a specific CDK (Current Opinion in Pharmacology, 2003(3): 362-370). Purine-based CDK inhibitors, such as olomoucine, roscovitine, purvanolols and CGP74514A are known to exhibit a greater selectivity for CDKs 1, 2 and 5, but show no inhibitory activity against CDKs 4 and 6 (Current Opinion in Pharmacology, 2003(3): 362-370). Furthermore, it has been demonstrated that purine-based CDK inhibitors such as roscovitine can exert anti-apoptotic effects in the nervous system (Pharmacol Ther 2002, 93:135-143) or prevent neuronal death in neurodegenerative diseases, such as Alzheimers's disease (Biochem Biophys Res Commun 2002 (297):1154-1158; Trends Pharmacol Sci 2002 (23):417-425).

While the use of CDK inhibitors has been described for a variety of therapeutic applications, there is only limited knowledge regarding the use of CDK inhibitors in the management of pain.

Current treatments for pain are only partially effective, and many also cause debilitating or dangerous side effects. For example, many of the traditional analgesics used to treat severe pain induce debilitating side effects such as nausea, dizziness, constipation, respiratory depression, and cognitive dysfunction (Brower, 2000).

Although there is already a broad panel of approved pain medications like non-narcotic analgesics, opioid analgesics, calcium channel blockers, muscle relaxants, and systemic corticosteroids available, said treatments remain merely empirical and, while they may relieve the symptoms of pain, they do not lead to complete relief in most cases. This is also due to fact that the mechanisms underlying the development of the different types of pain are still only poorly understood. Researchers are only just beginning to appreciate the complexity and diversity of the signaling systems used to relay nerve impulses for each type of pain.

Generally, pain is defined as an unpleasant sensory and emotional experience associated with actual or potential tissue damage, or described in terms of such damage, according to the International Association for the Study of Pain (IASP). Specifically, pain may occur as acute or chronic pain.

Acute pain occurs for brief periods of time, typically less than 1 month and is associated with temporary disorders. It is a natural body response to let the host be aware of physiological or biochemical alteration that could result in further damage within a short period of time. It is felt when noxious stimuli activate high threshold mechanical and/or thermal nociceptors in peripheral nerve endings and the evoked action potentials in thinly myelinated (Aδ) and/or unmyelinated (C) afferent fibres reach a conscious brain. Said noxious stimuli may be provided by injury, surgery, illness, trauma or painful medical procedures. Acute pain usually disappears when the underlying cause has been treated or has healed. Unrelieved acute pain, however, may lead to chronic pain problems that may result in long hospital stays, rehospitalizations, visits to outpatient clinics and emergency departments, and increased health care costs.

In contrast to acute pain, chronic pain persists long after the initial injury has healed and often spreads to other parts of the body, with diverse pathological and psychiatric consequences. Chronic somatic pain results from inflammatory responses to trauma in peripheral tissues (e.g., nerve entrapment, surgical procedures, cancer, or arthritis), which leads to oversensitization of nociceptors and intense responses of searing pain to normally non-noxious stimuli (hyperalgesia). Chronic pain is continuous and recurrent and its intensity will vary from mild to severe disabling pain that may significantly reduce quality of life.

Chronic pain is currently treated with conventional analgesics such as NSAIDs (Ibuprofen, Naproxen), Cox-2 inhibitors (Celecoxib, Valdecoxib, Rofecoxib) and opiates (codeine, morphin, thebain, papaverin, noscapin). For a significant number of patients however, these drugs provide insufficient pain relief.

Another subtype of pain, inflammatory pain, can occur as acute as well as chronic pain. Resulting injuries of tissue and neurons must not but may develop into long-lasting chronic neuropathic pain effects in succession to such inflammatory events.

Inflammatory pain is mediated by noxious stimuli like e.g. inflammatory mediators (e.g. cytokines, such as TNF α, prostaglandins, substance P, bradykinin, purines, histamine, and serotonine), which are released following tissue injury, disease, or inflammation and other noxious stimuli (e.g. thermal, mechanical, or chemical stimuli). In addition, cytokines and growth factors can influence neuronal phenotype and function (Besson 1999). These mediators are detected by nociceptors (sensory receptors) that are distributed throughout the periphery of the tissue. Said nociceptors are sensitive to noxious stimuli (e.g. mechanical, thermal, or chemical), which would damage tissue if prolonged (Koltzenburg 2000). A special class of so called C-nociceptors represent a class of “silent” nociceptors that do not respond to any level of mechanical or thermal stimuli but are activated in presence of inflammation only.

Current approaches for the treatment of especially inflammatory pain aim at cytokine inhibition (e.g. IL1β) and suppression of pro-inflammatory TNFα. Current approved anticytokine/antiTNFalpha treatments are based on chimeric antibodies such as Infliximab and Etanercept which reduce TNFα circulation in the bloodstream. TNFα is one of the most important inflammatory mediators that induce synthesis of important enzymes such as COX-2, MMP, iNOS, cPLa₂ and others. The main drawbacks of these “biologicals”, however, reside in their immunogenic potential with attendant loss of efficacy and their kinetics that lead to a more or less digital all- or -nothing reduction of circulating TNFalpha. The latter can result in severe immune suppressive side effects.

A distinct form of chronic pain, neuropathic (or neurogenic) pain, arises as a result of peripheral or central nerve dysfunction and includes a variety of conditions that differ in etiology as well as location. Generally, the causes of neuropathic pain are diverse, but share the common symptom of damage to the peripheral nerves or components of central pathways. The causative factors might be metabolic, viral or mechanical nerve lesion. Neuropathic pain is believed to be sustained by aberrant somatosensory processes in the peripheral nervous system, the CNS, or both. Neuropathic pain is not directly linked to stimulation of nociceptors, but instead, is thought to arise e.g. from oversensitization of glutamate receptors on postsynaptic neurons in the gray matter (dorsal horn) of the spinal cord.

Neuropathic pain is associated with conditions such as nerve degeneration in diabetes and postherpetic neuralgia (shingles). Neuropathic pain conditions are the consequence of a number of diseases and conditions, including diabetes, AIDS, multiple sclerosis, stump and phantom pain after amputation, cancer-related neuropathy, post-herpetic neuralgia, traumatic nerve injury, ischemic neuropathy, nerve compression, stroke, spinal cord injury.

Management of neuropathic pain remains a major clinical challenge, partly due to an inadequate understanding of the mechanisms involved in the development and maintenance of neuropathic pain. Many existing analgesics are ineffective in treating neuropathic pain and most of current narcotic and non-narcotic drugs do not control the pain. Current clinical practice includes the use of a number of drug classes for the management of neuropathic pain, for example anticonvulsants, tricyclic antidepressants, and systemic local anaesthetics. However, the usual outcome of such treatment is partial or unsatisfactory pain relief, and in some cases the adverse effects of these drugs outweigh their clinical usefulness. Classic analgesics are widely believed to be poorly effective or ineffective in the treatment of neuropathic pain. Few clinical studies on the use of non steroidal anti-inflammatory drugs (NSAIDs) or opiates in the treatment of neuropathic pain have been conducted, but in those which have, the results appear to indicate that NSAIDs are poorly effective or ineffective and opiates only work at high doses. A review analysing the controlled clinical data for peripheral neuropathic pain (PNP) (Pain, November, 1997 73(2), 123-39) reported that NSAIDs were probably ineffective as analgesics for PNP and that there was no long-term data supporting the analgesic effectiveness of any drug.

Available analgesic drugs often produce insufficient pain relief. Although tricyclic antidepressants and some antiepileptic drugs, for example gabapentin, lamotrigine and carbamazepine, are efficient in some patients, there remains a large unmet need for efficient drugs for the treatment of these conditions.

In conclusion, there is a need for safe and effective methods of pain treatment, in particular of chronic inflammatory and neuropathic pain.

This need is met by the present invention, which provides methods of treating pain comprising the administration of inhibitors of cyclin-dependent kinases to subjects in need thereof.

### SUMMARY OF THE INVENTION

The present invention is directed to inhibitors of cyclin-dependent kinases and to methods and compositions for treating and/or preventing any type of pain comprising: administering an effective amount of at least one inhibitor of a cyclin-dependent kinase (cdk, CDK) to a subject in need thereof.

In a preferred embodiment, the inhibitor of cyclin-dependent kinases inhibits cdk9. In another preferred embodiment, the inhibitor is selected among compounds described in WO2005/012262 according to general Formula I:

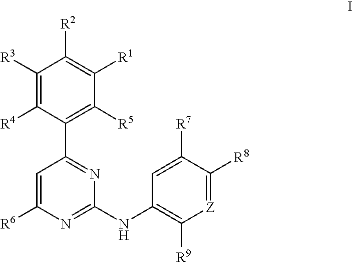

wherein:

### Z is CR¹⁰ or N;

one of R¹ and R² is selected from (CH₂)ₘR¹¹, (CH₂)ₘR¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)nR¹¹, (CH₂)ₘCONR¹²R¹³, (CH₂)ₘCO(CH₂)ₙR¹¹ and (CH₂)COR¹²;
where m is 0, 1, 2, 3 or 4, and n is 1, 2, 3 or 4; the other of R¹ and R² is H or R¹¹;
R³ and R⁵ are both H;

### R⁴ is H or R¹¹;

R⁶ is H or (CH₂)ₚR¹¹, where p is 0 or 1;
R⁷, R⁹ and R¹⁰ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;
each R¹¹ is independently halogen, NO₂, CN, (CH₂)_{q}OR¹³ (CH₂)ᵣNR¹³R¹⁴, NHCOR¹³, CF₃, COR¹³, R¹³, CONR¹³R¹⁴, SO₂NR¹³R¹⁴, SO₂R¹³, OR¹², NR¹³SO₂R¹⁴, OCH₂CH₂OH, OCH₂CH₂OMe, NR¹³SO₂R¹³, (CH₂)ₛNR¹²R¹³, morpholine, piperidine or piperazine, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
providing that:
when -Z is CR¹⁰ and R⁹ is H, then at least one of R⁷, R⁸ and R¹⁰ is other than OMe; and
when -Z is CR¹⁰ and R⁷, R⁸, and R⁹ are all H, then R¹⁰ is other than OCF₂CHF₂;
and the N-oxide derivatives, prodrug derivatives, protected derivatives, individual isomers, mixtures of isomers, enantiomers, diastereomers thereof; and the pharmaceutically acceptable salts and solvates (e.g., hydrates) of such compounds.

In a further embodiment, the invention provides the above-mentioned compounds according to any one of general Formula I for medical use.

In a further embodiment, the invention provides pharmaceutical compositions comprising a compound according to any one of general formula I as outlined above, together with a pharmaceutically acceptable carrier.

In a further embodiment, the invention provides the use of a compound according to any of general formula I as outlined above for preparing a pharmaceutical composition for treating any type of pain, chronic pain, inflammatory and/or neuropathic pain.

In a further embodiment, the invention provides a method of treating any type of pain, chronic pain, inflammatory and/or neuropathic pain comprising the administration of an effective amount of at least one of the compounds as mentioned above to a subject in need thereof.

Furthermore, the present invention relates to a method of inhibiting cdk9, said method comprising administering to a subject in need thereof an effective cdk9 inhibiting amount of at least one compound according to any one of general Formula I.

### BRIEF DESCRIPTION OF THE DRAWINGS

FIG. 1 schematically depicts the spared nerve injury model (SNI model, as developed by Decosterd and Woolf, 2000), which is characterized by ligation and section of two branches of the sciatic nerve (tibial and common peroneal nerves) leaving the sural nerve intact.

FIG. 2 schematically depicts the putative role of CDK9 as a target in the development of pain.

FIG. 3 shows reduction of LPS-induced cytokine levels by administration of compound #133 as exemplified for TNFα-levels.

FIG. 4 depicts reduction of LPS-induced cytokine levels of up to 100% by administration of compound #133 as exemplified for TNFα-, IL6-, and Il1β-levels.

### DETAILED DESCRIPTION OF THE INVENTION

The present invention is directed to inhibitors of cyclin-dependent kinases and to methods and compositions for treating and/or preventing any type of pain, comprising: administering an effective amount of at least one inhibitor of a cyclin-dependent kinase (cdk, CDK) to a subject in need thereof.

As used herein, the term “cyclin-dependent kinase inhibitor” refers to any compound, or group of compounds capable of downregulating, decreasing, suppressing or otherwise regulating the amount and/or activity of a cyclin-dependent kinase. Inhibition of said kinases can be achieved by any of a variety of mechanisms known in the art, including, but not limited to binding directly to the kinase polypeptide, denaturing or otherwise inactivating the kinase, or inhibiting the expression of the gene (e.g., transcription to mRNA, translation to a nascent polypeptide, and/or final polypeptide modifications to a mature protein), which encodes the kinase. Furthermore, a cyclin-dependent kinase inhibitor may also interfere with expression, modification, regulation or activation of any molecule acting downstream of a CDK in a CDK-dependent pathway.

In a preferred embodiment, kinase inhibitors according to this invention may be proteins, polypeptides, nucleic acids, small molecules, monoclonal or polyclonal antibodies directed against cyclin-dependent kinases, or other chemical moieties.

In a preferred embodiment of this invention, a cyclin-dependent kinase inhibitor according to the present invention inhibits a CDK selected from the group consisting of CDK1, CDK2, CDK3, CDK4, CDK5, CDK6, CDK7, CDK8, CDK9, CDK10, CDK11, CrkRS (Crk7, CDC2-related protein kinase 7), CDKL1 (cyclin-dependent kinase-like 1); KKIALRE, CDKL2 (cyclin-dependent kinase-like 2), KKIAMRE, CDKL3 (cyclin-dependent kinase-like 3), NKIAMRE, CDKL4, similar to cyclin-dependent kinase-like 1, CDC2L1 (cell division cycle 2-like 1), PITSLRE B, CDC2L1 (cell division cycle 2-like 1), PITSLRE A, CDC2L5 (cell division cycle 2-like 5), PCTK1 (PCTAIRE protein kinase 1), PCTK2 (PCTAIRE protein kinase 2), PCTK3 (PCTAIRE protein kinase 3) or PFTK1 (PFTAIRE protein kinase 1).

The inhibitor may also inhibit more than one cyclin-dependent kinase selected from the above-recited group.

In a further embodiment of this invention, the cyclin-dependent kinase inhibitor selectively inhibits one or more CDKs without having a substantial inhibitory effect on other enzymes or proteins.

In a particularly preferred embodiment of this invention, the cyclin-dependent kinase inhibitor inhibits CDK9.

In a preferred embodiment, such inhibitory compounds display an increased selectivity for a particular CDK. “Increased selectivity” as used herein means that the inhibitory compound is at least 10-100× more selective for a particular CDK selected from the group of CDKs as recited herein, supra. In a preferred embodiment of the present invention, the inhibitory compound is 20-90× more selective for a particular CDK. In a particular preferred embodiment, the inhibitory compound is 30-80× more selective for a particular CDK.

In a particular preferred embodiment, the cyclin-dependent kinase inhibitor has an increased selectivity for CDK9 than for other CDKs.

As used herein, the term “inhibiting” or “inhibition” refers to the ability of a compound to downregulate, decrease, reduce, suppress, inactivate, or inhibit at least partially the cellular function of a cyclin-dependent kinase, i.e. its activity or the expression of the cyclin-dependent kinase.

Suitable inhibitors are substituted pyrimidine derivatives such as [4-(3-substituted-phenyl)-pyrimidin-2-yl]-phenyl-amines and/or [4-(3-substituted-phenyl)-pyrimidin-2-yl]-(pyridine-3-yl)-amines as described in patent application WO 2005/012262. Thus, in a particularly preferred embodiment, the cyclin-dependent kinase inhibitor is selected among the compounds as described in WO 2005/012262 which are characterized by Formula I:

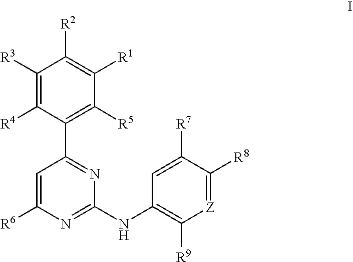

wherein:

### Z is CR¹⁰ or N;

one of R¹ and R² is selected from (CH₂)ₘR¹¹, (CH₂)ₘR¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)ₙR¹¹, (CH₂)ₘCONR¹²R¹³, (CH₂)ₘCO(CH₂)ₙR¹¹ and (CH₂)COR¹²;
where m is 0, 1, 2, 3 or 4, and n is 1, 2, 3 or 4;
the other of R¹ and R² is H or R¹¹;
R³ and R⁵ are both H;

### R⁴ is H or R¹;

R⁶ is H or (CH₂)ₚR¹¹, where p is 0 or 1;
R⁷, R⁹ and R¹⁰ are each independently H or R¹¹;

R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;

each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, OR¹², NR¹³SO₂R¹⁴ OCH₂CH₂OH, OCH₂CH₂OMe, NR¹³SO₂R¹³, (CH₂)ₛNR¹²R¹³, morpholine, piperidine or piperazine, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
providing that:
when -Z is CR¹⁰ and R⁹ is H, then at least one of R⁷, R⁸ and R¹⁰ is other than OMe; and
when -Z is CR¹⁰ and R⁷, R⁸, and R⁹ are all H, then R¹⁰ is other than OCF₂CHF₂;
and the N-oxide derivatives, prodrug derivatives, protected derivatives, individual isomers, mixtures of isomers, enantiomers, diastereomers thereof; and the pharmaceutically acceptable salts and solvates (e.g., hydrates) of such compounds.

In another preferred embodiment, the cyclin-dependent kinase inhibitor is selected among the compounds as described in WO 2005/012262 which are characterized by Formula Ia:

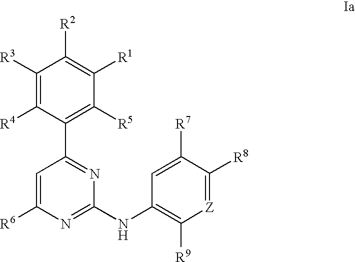

wherein:

### Z is CR¹⁰ or N;

R¹ is selected from (CH₂)ₘR¹¹, (CH₂)R¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)ₙR¹¹, (CH₂)ₘCONR¹²R¹³, (CH₂)ₘCO(CH₂)ₙR¹¹ and (CH₂)ₘCOR¹²; where m is 0, 1, 2, 3 or 4, and n is 1, 2, 3 or 4;
R³ and R⁵ are both H;
R² and R⁴ are each independently H or R¹¹;
R⁶ is H or (CH₂)ₚR¹¹, where p is 0 or 1;
R⁷, R⁹ and R¹¹ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴ SO₂R¹³, NR¹³SO₂R¹⁴, OCH₂CH₂OH, OCH₂CH₂OMe, morpholine, piperidine, and piperazine;
each R¹¹ is independently halogen, NO₂, CN, OR¹³, NR¹³R¹⁴COR¹³, CF₃, COR¹³, R¹³, CONR¹³R¹⁴, SO₂NR¹³R¹⁴, SO₂R¹³, OR¹³, NR¹³SO₂R¹⁴, OCH₂CH₂OH, OCH₂CH₂OMe, morpholine, piperidine or piperazine;
each R¹² is independently a hydrocarbyl group optionally containing one or more heteroatoms and optionally substituted with one or more R¹¹ groups;
each R¹³ and each R¹⁴ is independently H or an alkyl group;
and R¹⁵ is an alkyl group;
providing that:
when -Z is CR¹⁰ and R⁹ is H, then at least one of R⁷, R⁸ and R⁹ is other than OMe; and
when -Z is CR¹⁰ and R⁷, R⁸, and R⁹ are all H, then R¹⁰ is other than OCF₂CHF₂;
and the N-oxide derivatives, prodrug derivatives, protected derivatives, individual isomers, mixtures of isomers, enantiomers, diastereomers thereof; and the pharmaceutically acceptable salts and solvates (e.g., hydrates) of such compounds.

In one preferred embodiment, one of R¹ and R² is selected from (CH₂)ₘR¹¹, (CH₂)ₘR¹², (CH₂)ₘNR¹²R¹³, (CH₂)ₘNR¹³COR¹², and (CH₂)ₘOR¹².

In one preferred embodiment, R¹ is selected from (CH₂)ₘR¹¹, (CH₂)ₘR¹², (CH₂)ₘNR¹²R¹³, (CH₂)ₘNR¹³COR¹², and (CH₂)ₘOR¹².

In one preferred embodiment, one of R¹ and R² is selected from NO₂, CN, halogen, CH₂R¹¹, CH₂R, OR, NR¹²R, NR¹³COR, CH₂NRR, CH₂NHSO₂R, CF₃, NR¹³R¹⁴, R¹³, CH₂NR¹³COR¹² and NR¹³SO₂R¹².

In another preferred embodiment, R¹ is selected from NO₂, CN, halogen, CH₂R¹¹, CH₂R¹², OR¹², NR¹²R¹³, NR¹³COR¹², CH₂NR¹²R¹³, CH₂NHSO₂R¹⁴, CF₃, NR¹³R¹⁴, R¹³, CH₂NR¹³COR¹² and NR¹³SO₂R¹².

In one particularly preferred embodiment, R¹ is selected from NO₂, CN, halogen, (CH₂)ₘR¹¹, (CH₂)ₘR¹², (CH₂)ₘNR¹²R¹³, (CH₂)ₘNR¹³COR¹², and (CH₂)ₘOR¹².

In another preferred embodiment, R¹ is selected from NO₂, CN, halogen, CH₂R¹¹, CH₂R¹², OR¹²NR¹²R¹³, NR¹³COR¹², CH₂NR¹²R¹³ and CH₂NHSO₂R¹⁴.

In one preferred embodiment, R⁴ is H, OR¹³, halogen or R¹³.

In a more preferred embodiment, R⁴ is H, OMe, Me or F.

In one particularly preferred embodiment, each R¹² is independently selected from alkyl, alkenyl, alkynyl, aralkyl, a cyclic group, a saturated or unsaturated alicyclic group, and an aryl group, each of which may optionally contain one to four heteroatoms selected from O, S, and N, and each of which may optionally be substituted with one, two or three R¹¹ groups.

In one particularly preferred embodiment, each R¹² is independently selected from alkyl, alkenyl, alkynyl, aralkyl, a heteroaryl group, a saturated or unsaturated alicyclic group optionally contain one to four heteroatoms selected from O, S, and N, and an aryl group, each of which may optionally be substituted with one, two or three R¹¹ groups.

In one preferred embodiment, R¹² is selected from aryl, aralkyl heteroaryl and a saturated alicyclic group optionally contain one to four heteroatoms selected from O, S, and N, each of which may optionally be substituted with one, two or three R¹¹ groups.

In a more preferred embodiment, R¹² is selected from phenyl, benzyl, 1,2,4-triazolyl, N-piperidinyl, N-morpholino, N-pyrrolidinyl and N-piperidinyl, each of which may optionally be substituted with one, two or three R¹¹ groups.

In an even more preferred embodiment, R¹² is selected from phenyl, benzyl, 1,2,4-triazolyl, N-piperidinyl, N-morpholino, N-pyrrolidinyl and N-piperidinyl, each of which may optionally be substituted with one, two or three substituents selected from NO₂, CON¹³R¹⁴, (CH₂)_{q}OR¹³ and R¹³.

In a further preferred embodiment, R¹² is selected from phenyl, benzyl, 1,2,4-triazolyl, N-piperidinyl, N-morpholino, N-pyrrolidinyl and N-piperidinyl, each of which may optionally be substituted with one, two or three substituents selected from NO₂, CONH₂, CH₂CH₂OH, CH₂OH and Me groups.

Preferably, R¹⁵ is a C₁₋₅ alkyl group.

Preferably, each R¹³ and each R¹⁴ is independently H or a C₁₋₅ alkyl group.

Even more preferably, each R¹³ and R¹⁴ is independently H or an unsubstituted C₁₋₅ alkyl group.

In one especially preferred embodiment, each R¹² is independently selected from alkyl, alkenyl, alkynyl, aralkyl, a cyclic group, a saturated or unsaturated alicyclic group, and an aryl group, each of which may optionally contain one to four heteroatoms selected from O, S, and N, and each of which may optionally be substituted with one, two or three R¹¹ groups; each R¹³ and each R¹⁴ is independently H or a C₁₋₅ alkyl group; and R¹⁵ is a C₁₋₅ alkyl group.

Preferably, R¹⁵ is an unsubstituted C₁₋₅ alkyl group.

In one preferred embodiment, each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, NR¹³SO₂R¹⁴, OCH₂CH₂OH, OCH₂CH₂OMe, NR¹³SO₂R¹², (CH₂)ₛNR¹²R¹³, morpholino, piperidinyl or piperazinyl, where q, r and s are each independently 0, 1, 2, 3 or 4.

In another preferred embodiment, each R¹¹ is selected from halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl and piperazinyl.

In another especially preferred embodiment, R¹¹ is selected from halogen, NO₂, CN, OH, NH₂, NHCOMe, CF₃, COMe, Me, Et, ⁱPr, NHMe, NMe₂, CONH₂, CONHMe, CONMe₂, SO₂NH₂, SO₂NHMe, SO₂NMe₂, SO₂Me, OMe, OEt, OCH₂CH₂OH, OCH₂CH₂OMe, morpholino, piperidinyl and piperazinyl.

In a preferred embodiment, one of R¹ and R² is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr) (Et), NHEt, CH₂NHCH₂Ph, NHEt, Me, CH₂NMe₂, OH, CF₃, NMeSO₂Me, CH₂N(ⁱPr)COMe, CH₂OH, CH₂NEt₂

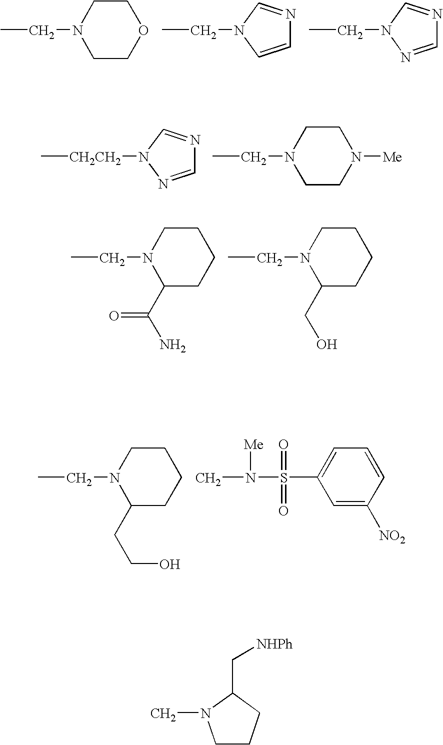

In a more preferred embodiment, R¹ is selected from NO₂, NH₂, N(Et)COMe, NHCOMe, N(Me)COMe, N(ⁱPr)COMe, NHMe, Cl, F, CN, CH₂NHSO₂Me, OMe, CH₂N(ⁱPr)(Et), NHEt, CH₂NHCH₂Ph, NHEt, Me, CH₂NMe₂, OH, CF₃, NMeSO₂Me, CH₂N(ⁱPr)COMe, CH₂OH, CH₂NEt₂,

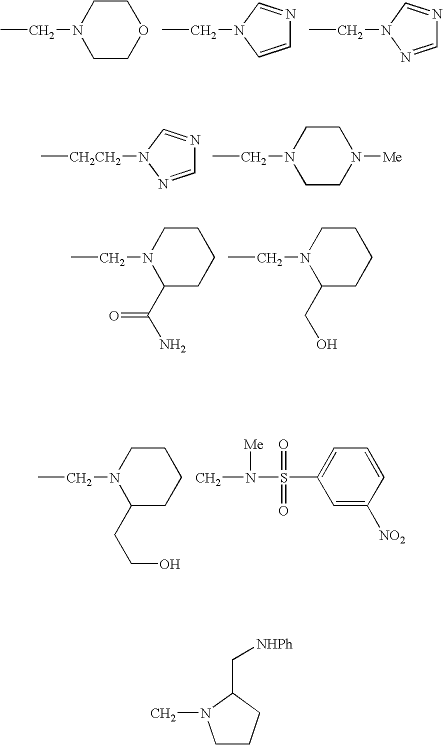

In one preferred embodiment, R² is H, halogen, OR¹³ or (CH₂)ₘR¹².

Even more preferably, R² is selected from H, Cl, OMe, OEt,

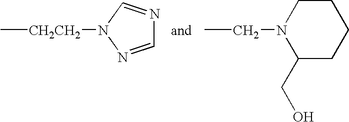

In one particularly preferred embodiment, R¹ is selected from NO₂, NH₂, N(Et)COMe, NHCOMe, N(Me)COMe, N(ⁱPr)COMe, NHMe, Cl, F, CN, CH₂NHSO₂Me, OMe, CH₂Ni(Pr)(Et)NHEt, CH₂NHCH₂Ph,

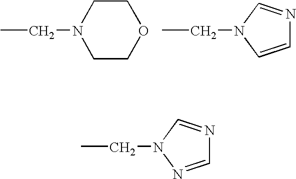

In one preferred embodiment, R⁷, R⁸, R⁹, and R¹⁰ are each independently selected from H, halogen, NO₂, CN, OH, NH₂, NHCOMe, CF₃, COMe, Me, Et, ⁱPr, NHMe, NMe₂, CONHMe, CONMe₂, SO₂NH₂, SO₂NHMe, SO₂NMe₂, SO₂Me, OMe, OEt, OCH₂CH₂OH, OCH₂CH₂OMe, CH₂OH, morpholino, piperidinyl, and piperazinyl.

In one preferred embodiment, R⁶ and R⁹ are both H.

In one preferred embodiment, R⁷ is selected from H, NO₂, NR¹³R¹⁴, OR¹³, CN, CF₃, CH²OR¹³, SO₂R¹³, and halogen.

In a more preferred embodiment, R⁷ is selected from H, NO₂, NH₂, OH, OMe, CN, CH₂OH, CF₃, and SO₂Me.

In a more preferred embodiment, R⁸ is selected from H, OH, NO₂, OCH₂CH₂OMe, Cl, F, NMe₂, N-morpholino, Me and OMe.

In another particularly preferred embodiment, R⁷, R⁸, R⁹, and R¹⁰ are each independently selected from H, halogen, NO₂, CN, OH, NH₂, NHCOMe, CF₃, COMe, Me, Et, ⁱPr, NHMe, NMe₂, CONHMe, CONMe₂, SO₂NH, SO₂NHMe, SO₂NMe₂, SO₂Me, OMe, OEt, OCH₂CH₂OH, OCH₂CH₂OMe, morpholino, piperidinyl, and piperazinyl.

Preferably, R⁷, R⁸ and R⁹ are each independently selected from H, halogen, NO₂, CN, OR¹³, NR¹³R¹⁴, NHCOR¹³, CF3, COR¹³, R¹³, CONR¹³R¹⁴, SO₂NR¹³R¹⁴, SO₂R¹³, OR¹³, NR¹³SO₂R¹⁴, OCH₂CH₂OH, OCH₂CH₂OMe, morpholino, piperidinyl and piperazinyl.

Preferably, R² is H or halogen; R⁴ is H or OR¹³; R⁶ and R⁹ are both H; R⁷ is selected from H, NO₂, NR¹³R¹⁴, OR¹³ and CN; R⁸ is selected from H, OR¹³, NO₂, OCH₂CH₂OMe, halogen, NR¹³R¹⁴, N-morpholino and OMe.

Preferably, where Z is CR and R⁹ is H, at least two of R⁷, R⁸ and R¹⁰ are other than OMe.

In yet another particularly preferred embodiment, R² is H or Cl; R¹⁴ is H or OMe; R⁷ is selected from H, NO₂, NH₂, OH, OMe and CN; and R⁸ is selected from H, OH, NO₂, OCH₂CH₂OMe, Cl, F, NMe₂, and N-morpholino.

In one preferred embodiment, Z is CR¹⁰.

Preferably, R¹⁰ is selected from H, halogen, NO₂, CN, OR¹³, NR¹³R¹⁴, NHCOR¹³, CF₃, COR¹³, R¹³, CONR¹³R¹⁴, SO₂NR¹³R¹⁴, SO₂R¹³, NR¹³SO₂R¹⁴, OCH₂CH₂OH, OCH₂CH₂OMe, morpholino, piperidinyl and piperazinyl.

More preferably, R¹⁰ is selected from NO₂, NH₂, H, OH, OMe, CN, F, CH₂OH, CF₂ and SO₂Me.

More preferably still, R¹⁰ is H.

In another preferred embodiment, Z is N.

As used herein, the term “hydrocarbyl” refers to a group comprising at least C and H. If the hydrocarbyl group comprises more than one C, then those carbons need not necessarily be linked to each other. For example, at least two of the carbons may be linked via a suitable element or group. Thus, the hydrocarbyl group may contain heteroatoms. Suitable heteroatoms will be apparent to those skilled in the art and include, for instance, sulphur, nitrogen, oxygen, phosphorus and silicon. Where the hydrocarbyl group contains one or more heteroatoms, the group may be linked via a carbon atom or via a heteroatom to another group, i.e. the linker atom may be a carbon or a heteroatom. Preferably, the hydrocarbyl group is an aryl, heteroaryl, alkyl, cycloalkyl, aralkyl, alicyclic, heteroalicyclic or alkenyl group. More preferably, the hydrocarbyl group is an aryl, heteroaryl, alkyl, cycloalkyl, aralkyl or alkenyl group. The hydrocarbyl group may be optionally substituted by one or more R¹¹ groups.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group.

Particularly preferred alkyl groups include, for example, methyl, ethyl, propyl, isopropyl (ⁱPr), butyl, isobutyl, tert-butyl, pentyl and hexyl. Suitable substituents include, for example, one or more R¹¹ groups. Preferably, the alkyl group is unsubstituted.

As used herein, the term “cycloalkyl” refers to a cyclic alkyl group which may be substituted (mono- or poly-) or unsubstituted. Preferably, the cycloalkyl group is a C₃₋₁₂ cycloalkyl group. Suitable substituents include, for example, one or more R¹¹ groups.

As used herein, the term “alkenyl” refers to a group containing one or more carbon-carbon double bonds, which may be branched or unbranched, substituted (mono- or poly-) or unsubstituted. Preferably the alkenyl group is a C₂₋₂₀ alkenyl group, more preferably a C₂₋₁₅ alkenyl group, more preferably still a C₂₋₁₂ alkenyl group, or preferably a C₂₋₅ alkenyl group, more preferably a C₂₋₃ alkenyl group. Suitable substituents include, for example, one or more R¹¹ groups as defined above.

As used herein, the term “aryl” refers to a C₆₋₁₂ aromatic group which may be substituted (mono- or poly-) or unsubstituted. Typical examples include phenyl and naphthyl etc.

Suitable substituents include, for example, one or more R¹¹ groups.

As used herein, the term “heteroaryl” refers to a C₂₋₁₂ aromatic, substituted (mono- or poly-) or unsubstituted group, which comprises one or more heteroatoms. Preferably, the heteroaryl group is a C₄₋₁₂ aromatic group comprising one or more heteroatoms selected from N, O and S. Suitable heteroaryl groups include pyrrole, pyrazole, pyrimidine, pyrazine, pyridine, quinoline, thiophene, 1,2,3-triazole, 1,2,4-triazole, thiazole, oxazole, iso-thiazole, iso-oxazole, imidazole, furan and the like. Again, suitable substituents include, for example, one or more R¹¹ groups.

As used herein, the term “alicyclic” refers to a cyclic aliphatic group which optionally contains one or more heteroatoms. Preferred alicyclic groups include piperidinyl, pyrrolidinyl, piperazinyl and morpholino. More preferably, the alicyclic group is selected from N-piperidinyl, N-pyrrolidinyl, N-piperazinyl and N-morpholino.

As used herein, the term “aralkyl” includes, but is not limited to, a group having both aryl and alkyl functionalities. By way of example, the term includes groups in which one of the hydrogen atoms of the alkyl group is replaced by an aryl group, e.g. a phenyl group optionally having one or more substituents such as halo, alkyl, alkoxy, hydroxy, and the like. Typical aralkyl groups include benzyl, phenethyl and the like.

In one preferred embodiment of this invention, the cyclin-dependent kinase inhibitor is selected among the following compounds as described in WO 2005/012262:
4-[4-(3-Nitro-phenyl)-pyrimidin-2-ylamino]-phenol [1]
(4-Nitro-phenyl)-[4-(3-nitro-phenyl)-pyrimidin-2-yl]-amine [2]
[4-(3-Amino-phenyl)-pyrimidin-2-yl]-[4-(2-methoxy-ethoxy)-phenyl]-amine [3]
[4-(3-Amino-phenyl)-pyrimidin-2-yl]-(4-nitro-phenyl)-amine [4]
(3-Nitro-phenyl)-[4-(3-nitro-phenyl)-pyrimidin-2-yl]-amine [5]
(4-Fluoro-phenyl)-[4-(3-nitro-phenyl)-pyrimidin-2-yl]-amine [6]
[4-(3-Amino-phenyl)-pyrimidin-2-yl]-(4-fluoro-phenyl)-amine [7]
N-[4-(3-Amino-phenyl)-pyrimidin-2-yl]-benzene-1,3-diamine [8]
N,N-Dimethyl-N′-[4-(3-nitro-phenyl)-pyrimidin-2-yl]-benzene-1,4-diamine [9]
N-Ethyl-N-{3-[2-(4-hydroxy-phenylamino)-pyrimidin-4-yl]-phenyl}-acetamide [10]
N-{3-[2-(4-Hydroxy-phenylamino)-pyrimidin-4-yl]-phenyl}-acetamide [11]
N-{3-[2-(4-Hydroxy-phenylamino)-pyrimidin-4-yl]-phenyl}-N-methyl-acetamide [12]
N-{3-[2-(4-Hydroxy-phenylamino)-pyrimidin-4-yl]-phenyl}-N-isobutyl-acetamide [13]
4-[4-(3-Methylamino-phenyl)-pyrimidin-2-ylamino]-phenol [14]
4-[4-(3-Amino-phenyl)-pyrimidin-2-ylamino]-phenol [15]
(4-Chloro-phenyl)-[4-(3-chloro-phenyl)-pyrimidin-2-yl]-amine [16]
4-[4-(3-Chloro-phenyl)-pyrimidin-2-ylamino]-phenol [17]
3-[4-(3-Chloro-phenyl)-pyrimidin-2-ylamino]-phenol [18]
[4-(3-Amino-phenyl)-pyrimidin-2-yl]-(3-nitro-phenyl)-amine [19]
N-[4-(3,4-Dichloro-phenyl)-pyrimidin-2-yl]-N′,N′-dimethyl-benzene-1,4-diamine [20]
4-[4-(3,4-Dichloro-phenyl)-pyrimidin-2-ylamino]-phenol [21]
3-[4-(3,4-Dichloro-phenyl)-pyrimidin-2-ylamino]-phenol [22]
N-Ethyl-N-{3-[2-(4-methoxy-phenylamino)-pyrimidin-4-yl]-phenyl}-acetamide [23]
N-Ethyl-N-{3-[2-(4-nitro-phenylamino)-pyrimidin-4-yl]-phenyl}-acetamide [24]
[4-(3-Ethylamino-phenyl)-pyrimidin-2-yl]-(4-methoxy-phenyl)-amine [25]
[4-(3-Ethylamino-phenyl)-pyrimidin-2-yl]-(4-nitro-phenyl)-amine [26]
{4-[3-(Benzylamino-methyl)-phenyl]-pyrimidin-2-yl}-(3-nitro-phenyl)=amine [27]
3-{4-[3-(Benzylamino-methyl)-phenyl]-pyrimidin-2-ylamino}-phenol [28]
[4-(3-Imidazol-1-ylmethyl-phenyl)-pyrimidin-2-yl]-(3-nitro-phenyl)-amine [29]
(3-Nitro-phenyl)-[4-(3-[1,2,4]triazol-1-ylmethyl-phenyl)-pyrimidin-2-yl]-amine [30]
[4-(3,4-Dichloro-phenyl)-pyrimidin-2-yl]-(3-nitro-phenyl)-amine [31]
(4-Morpholin-4-yl-phenyl)-[4-(3-[1,2,3]triazol-1-ylmethyl-phenyl)-pyrimidin-2-yl]-amine [32]
4-[4-(3-[1,2,4]Triazol-1-ylmethyl-phenyl)-pyrimidin-2-ylamino]-phenol [33]
3-[4-(3-[1,2,4]Triazol-1-ylmethyl-phenyl)-pyrimidin-2-ylamino]-phenol [34]
(3-Methoxy-phenyl)-[4-(3-[1,2,4]triazol-1-ylmethyl-phenyl)-pyrimidin-2-yl]-amine [35]
3-[4-(3-[1,2,4]Triazol-1-ylmethyl-phenyl)-pyrimidin-2-ylamino]-benzonitrile [36]Phenyl-(4-phenyl-pyrimidin-2-yl)-amine [37]
[4-(5-Fluoro-2-methoxy-phenyl)-pyrimidin-2-yl]-phenyl-amine [38]
[4-(3-Morpholin-4-ylmethyl-phenyl)-pyrimidin-2-yl]-(3-nitro-phenyl)-amine [39]
N-{3-[2-(3-Nitro-phenylamino)-pyrimidin-4-yl]-benzyl}-methanesulfonamide [40]
(4-Nitro-phenyl)-[4-(3-[1,2,4]triazol-1-ylmethyl-phenyl)-pyrimidin-2-yl]-amine [41]
(4-Methoxy-phenyl)-[4-(3-[1,2,4]triazol-1-ylmethyl-phenyl)-pyrimidin-2-yl]-amine [42]
N,N-Dimethyl-N′-[4-(3-[1,2,4]triazol-1-ylmethyl-phenyl)-pyrimidin-2-yl]-benzene-1,4-diamine [43]
[4-(2,5-Dimethoxy-phenyl)-pyrimidin-2-yl]-(3-nitro-phenyl)-amine [44]
4-[4-(2,5-Dimethoxy-phenyl)-pyrimidin-2-ylamino]-phenol [45]
(4-{3-[(Ethyl-isopropyl-amino)-methyl]-phenyl}-pyrimidin-2-yl)-(3-nitro-phenyl)-amine [46]
[4-(4-Chloro-3-[1,2,4]triazol-1-ylmethyl-phenyl)-pyrimidin-2-yl]-(3-nitro-phenyl)-amine [47]
{4-[3-(Benzylamino-methyl)-phenyl]-pyrimidin-2-yl}-(6-chloro-pyridin-3-yl-amine [48]
[4-(3,4-Dichloro-phenyl)-pyrimidin-2-yl]-(6-methoxy-pyridin-3-yl)-amine
(6-Methoxy-pyridin-3-yl)-[4-(3-[1,2,4]triazol-1-ylmethyl-phenyl)-pyrimidin-2-yl]-amine [50]
3-[2-(6-Methoxy-pyridin-3-ylamino)-pyrimidin-4-yl]-benzonitrile [51]
[4-(2,5-Dimethoxy-phenyl)-pyrimidin-2-yl]-(6-methoxy-pyridin-3-yl)-amine [52]
(4-3-[(Ethyl-isopropyl-amino)-methyl]-phenyl}-pyrimidin-2-yl)-(6-methoxy-pyridin-3-yl)-amine [53]
{4-[3-(4-Methyl-piperazin-1-ylmethyl)-phenyl]-pyrimidin-2-yl}-(3-nitro-phenyl)-amine [54]
3-[2-(3-Nitro-phenylamino)-pyrimidin-4-yl]-phenol [55]
3-[2-(3-Hydroxy-phenylamino)-pyrimidin-4-yl]-phenol [56]
3-[2-(3-Fluoro-phenylamino)-pyrimidin-4-yl]-phenol [57]
(6-Methoxy-pyridin-3-yl)-{4-[3-(4-methyl-piperazin-1-ylmethyl)-phenyl]-pyrimidin-2-yl}-amine [58]
[4-(3-Imidazol-1-ylmethyl-phenyl)-pyrimidin-2-yl]-(6-methoxy-pyridin-3-yl)-amine [59]
N-{3-[2-(3-Hydroxymethyl-phenylamino)-pyrimidin-4-yl]-phenyl}-acetamide [60]
[4-(2,5-Dimethyl-phenyl)-pyrimidin-2-yl]-(3-nitro-phenyl)-amine [61]
3-[4-(2,5-Dimethyl-phenyl)-pyrimidin-2-ylamino]-phenol [62]
[4-(2,5-Dimethyl-phenyl)-pyrimidin-2-yl]-(3-fluoro-phenyl)-amine [63]
3-[4-(3-Nitro-phenyl)-pyrimidin-2-ylamino]-phenol [64]
(3-Fluoro-phenyl)-[4-(3-nitro-phenyl)-pyrimidin-2-yl]-amine [65]
N-[3-(2-Phenylamino-pyrimidin-4-yl)-phenyl]-acetamide [66]
N-{3-[2-(3-Hydroxy-phenylamino)-pyrimidin-4-yl]-phenyl}-acetamide [67]
N-{3-[2-(3,5-Dimethoxy-phenylamino)-pyrimidin-4-yl]-phenyl}-acetamide [68]
N-{3-[2-(3-Nitro-phenylamino)-pyrimidin-4-yl]-phenyl}-acetamide [69]
N-{3-[2-(Pyridin-3-ylamino)-pyrimidin-4-yl]-phenyl}-acetamide [70]
[4-(3-Dimethylaminomethyl-phenyl)-pyrimidin-2-yl]-(3-nitro-phenyl)-amine [71]
3-[2 (3-Hydroxymethyl-phenylamino)-pyrimidin-4-yl]-phenol [72]
3-[2-(Pyridin-3-ylamino)-pyrimidin-4-yl]-phenol [73]
3-[2-(6-Methoxy-pyridin-3-ylamino)-pyrimidin-4-yl]-phenol [74]
3-[2-(3,5-Bis-trifluoromethyl-phenylamino)-pyrimidin-4-yl]-phenol [75]
3-[4-(4-Methoxy-phenyl)-pyrimidin-2-ylamino]-phenol [76]
[4-(3-Methoxy-phenyl)-pyrimidin-2-yl]-(6-methoxy-pyridin-3-yl)-amine [77]
N-Isopropyl-N-{3-[2-(3-nitro-phenylamino)-pyrimidin-4-yl]-benzyl}-acetamide [78]
(1-{3-[2-(3-Nitro-phenylamino)-pyrimidin-4-yl]-benzyl}-piperidin-2-yl)-methanol [79]
3-[4-(3-Dimethylaminomethyl-phenyl)-pyrimidin-2-ylamino]-phenol [80]
4-[4-(3-Dimethylaminomethyl-phenyl)-pyrimidin-2-ylamino]-phenol [81]
[4-(3-Dimethylaminomethyl-phenyl)-pyrimidin-2-yl]-(4-morpholin-4-yl-phenyl)-amine [82]
[4-(3-Dimethylaminomethyl-phenyl)-pyrimidin-2-yl]-(6-methoxy-pyridin-3-yl)-amine [83]
[4-(3-Diethylaminomethyl-phenyl)-pyrimidin-2-yl]-(3-nitro-phenyl)-amine [84]
N-Methyl-3-nitro-N-{3-[2-(3-nitro-phenylamino)-pyrimidin-4-yl]-benzyl}-benzenesulfonamide [85]
(3-Nitro-phenyl)-{4-[3-(2-phenylaminomethyl-pyrrolidin-1-ylmethyl)-phenyl]-pyrimidin-2-yl}-amine [86]
[4-(3-Methoxy-phenyl)-pyrimidin-2-yl]-(3-nitro-phenyl)-amine [87]
3-[4-(3-Methoxy-phenyl)-pyrimidin-2-ylamino]-phenol [88]
4-[4-(3,4-Dimethoxy-phenyl)-pyrimidin-2-ylamino]-phenol [89]
[4-(3,4-Dimethoxy-phenyl)-pyrimidin-2-yl]-(3-nitro-phenyl)-amine [90]
{3-[2-(3-Nitro-phenylamino)-pyrimidin-4-yl]-phenyl}-methanol [91]
3-[2-(3-Hydroxy-phenylamino)-pyrimidin-4-yl]-benzonitrile [92]
3-[2-(4-Hydroxy-phenylamino)-pyrimidin-4-yl]-benzonitrile [93]
[4-(4-Methoxy-phenyl)-pyrimidin-2-yl]-(3-nitro-phenyl)-amine [94]
3-[4-(3-Trifluoromethyl-phenyl)-pyrimidin-2-ylamino]-phenol [95]
4-[4-(3-Trifluoromethyl-phenyl)-pyrimidin-2-ylamino]-phenol [96]
(3-Nitro-phenyl)-[4-(3-trifluoromethyl-phenyl)-pyrimidin-2-yl]-amine [97]
4-[4-(3-Methoxy-phenyl)-pyrimidin-2-ylamino]-phenol [98]
1-{3-[2-(3-Nitro-phenylamino)-pyrimidin-4-yl]-benzyl}-piperidine-3-carboxylic acid amide [99]
2-(1-3-[2-(3-Nitro-phenylamino)-pyrimidin-4-yl]-benzyl}-piperidin-3-yl)-ethanol [100]
(1-{3-[2-(4-Morpholin-4-yl-phenylamino)-pyrimidin-4-yl]-benzyl}-piperidin-2-yl)-methanol [101]
(1-{3-[2-(6-Methoxy-pyridin-3-ylamino)-pyrimidin-4-yl]-benzyl}-piperidin-2-yl)-methanol [102]
3-4-[3-(2-Hydroxymethyl-piperidin-1-ylmethyl)-phenyl]-pyrimidin-2-ylamino}-phenol [103]
(3-Methanesulfonyl-phenyl)-[4-(3-[1,2,4]triazol-1-ylmethyl-phenyl)-pyrimidin-2-yl]-amine [104]
(1-{3-[2-(3-Nitro-phenylamino)-pyrimidin-4-yl]-benzyl}-piperidin-3-yl)-methanol [105]
4-{4-[3-(2-Hydroxymethyl-piperidin-1-ylmethyl)-phenyl]-pyrimidin-2-ylamino}-phenol [106]
(1-{3-[2-(3,5-Bis-hydroxymethyl-phenylamino)-pyrimidin-4-yl]-benzyl}-piperidin-2-yl)-methanol [107]
(1-3-[2-(4-Methyl-3-nitro-phenylamino)-pyrimidin-4-yl]-benzyl}-piperidin-2-yl)-methanol [108]
3-[4-(4-Ethoxy-phenyl)-pyrimidin-2-ylamino]-phenol [109]
4-[4-(4-Methoxy-phenyl)-pyrimidin-2-ylamino]-phenol [110]
[4-(4-Methoxy-phenyl)-pyrimidin-2-yl]-(4-morpholin-4-yl-phenyl)-amine [111]
[4-(3-Chloro-phenyl)-pyrimidin-2-yl]-(3-nitro-phenyl)-amine [112]
4-[4-(3-Fluoro-phenyl)-pyrimidin-2-ylamino]-phenol [113]
3-[4-(2,5-Difluoro-phenyl)-pyrimidin-2-ylamino]-phenol [114]
3-[4-(3-Hydroxymethyl-phenyl)-pyrimidin-2-ylamino]-phenol [115]
{3-[2-(3-Fluoro-phenylamino)-pyrimidin-4-yl]-phenyl}-methanol [116]
{3-[2-(3,5-Dinitro-phenylamino)-pyrimidin-4-yl]-phenyl}-methanol [117]
(3-Fluoro-phenyl)-[4-(3-methoxy-phenyl)-pyrimidin-2-yl]-amine [118]
(3-Fluoro-phenyl)-[4-(4-methoxy-phenyl)-pyrimidin-2-yl]-amine [119]
3-[2-(3,4,5-Trimethoxy-phenylamino)-pyrimidin-4-yl]-phenol [120]
3-[2-(3,5-Dimethoxy-phenylamino)-pyrimidin-4-yl]-phenol [121]
3-[2-(4-Hydroxy-phenylamino)-pyrimidin-4-yl]-phenol [122]
[4-(2,5-Difluoro-phenyl)-pyrimidin-2-yl]-(3-nitro-phenyl)-amine [123]
[4-(4-Methoxy-phenyl)-pyrimidin-2-yl]-(6-methoxy-pyridin-3-yl)-amine [124]
{3-[2-(6-Methoxy-pyridin-3-ylamino)-pyrimidin-4-yl]-phenyl}-methanol [125]
(3-Nitro-phenyl)-f4-[4-(2-[1,2,4]triazol-1-yl-ethyl)-phenyl]-pyrimidin-2-yl}-amine [126]
(1-{4-[2-(3-Nitro-phenylamino)-pyrimidin-4-yl]-benzyl}-piperidin-2-yl)-methanol [127]
[4-(4-Methoxy-phenyl)-pyrimidin-2-yl]-(3,4,5-trimethoxy-phenyl)-amine [128]
N-Methyl-N-{3-[2-(3-nitro-phenylamino)-pyrimidin-4-yl]-phenyl}-methanesulfonamide [129]
N-{3-[2-(3-Hydroxy-phenylamino)-pyrimidin-4-yl]-phenyl}-N-methyl-methanesulfonamide [130]
N-{3-[2-(4-Hydroxy-phenylamino)-pyrimidin-4-yl]-phenyl}-N-methyl-methanesulfonamide [131] and
N-{3-[2-(6-Methoxy-pyridin-3-ylamino)-pyrimidin-4-yl]-phenyl}-N-methyl-methanesulfonamide [132].

In a particularly preferred embodiment, the cyclin-dependent kinase inhibitor is selected among the following compounds of WO 2005/012262:

| Compound | | |
|---|---|---|
| No. | Compound | |
| 3 | 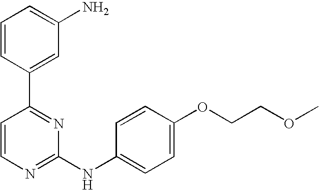 | [4-(3-Amino-phenyl)-pyrimidin-2-yl]-[4-(2-methoxy-ethoxy)-phenyl]-amine |
| 23 | 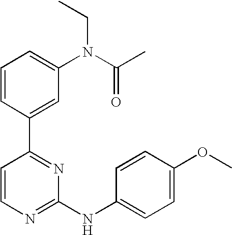 | N-Ethyl-N-{3-[2-(4-methoxy-phenylamino)-pyrimidin-4-yl]-phenyl}-acetamide |
| 25 | 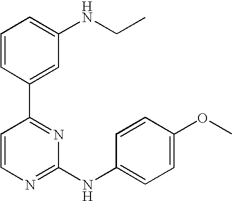 | [4-(3-Ethylamino-phenyl)-pyrimidin-2-yl]-(4-methoxy-phenyl)-amine |
| 26 | 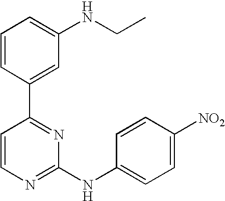 | [4-(3-Ethylamino-phenyl)-pyrimidin-2-yl]-(4-nitro-phenyl)-amine |
| 28 | 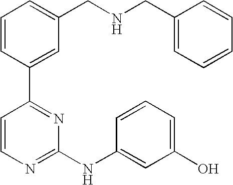 | 3-(4-[3-(Benzylamino-methyl)-phenyl]-pyrimidin-2-ylamino}-phenol |
| 29 | 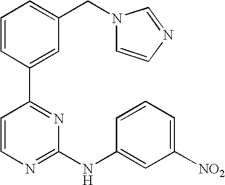 | [4-(3-Imidazol-1-ylmethyl-phenyl)-pyrimidin-2-yl]-(3-nitro-phenyl)-amine |
| 30 | 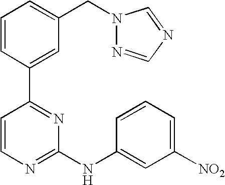 | (3-Nitro-phenyl)-[4-(3-[1,2,4]triazol-1-ylmethyl-phenyl)-pyrimidin-2-yl]-amine |
| 32 | 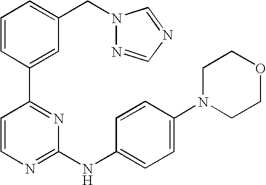 | (4-Morpholin-4-yl-phenyl)-[4-(3-[1,2,4]triazol-1-ylmethyl-phenyl)-pyrimidin-2-yl]-amine |
| 33 | 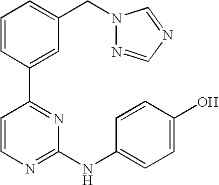 | 4-[4-(3-[1,2,4]Triazol-1-ylmethyl-phenyl)-pyrimidin-2-ylamino]-phenol |
| 34 | 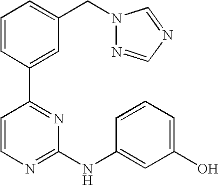 | 3-[4-(3-[1,2,4]Triazol-1-ylmethyl-phenyl)-pyrimidin-2-ylamino]-phenol |
| 35 | 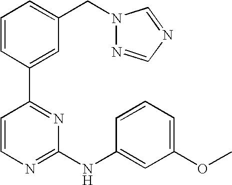 | (3-Methoxy-phenyl)-[4-(3-[1,2,4]triazol-1-ylmethyl-phenyl)-pyrimidin-2-yl]-amine |
| 36 | 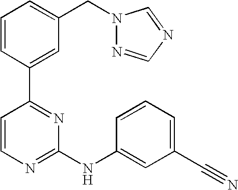 | 3-[4-(3-[1,2,4]Triazol-1-ylmethyl-phenyl)-pyrimidin-2-ylamino]-benzonitrile |
| 39 | 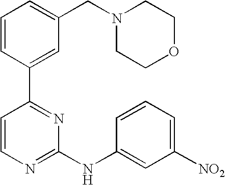 | [4-(3-Morpholin-4-ylmethyl-phenyl)-pyrimidin-2-yl]-(3-nitro-phenyl)-amine |
| 41 | 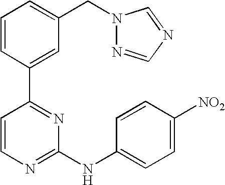 | (4-Nitro-phenyl)-[4-(3-[1,2,4]triazol-1-ylmethyl-phenyl)-pyrimidin-2-yl]-amine |
| 42 | 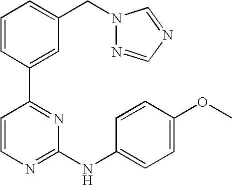 | (4-Methoxy-phenyl)-[4-(3-[1,2,4]triazol-1-ylmethyl-phenyl)-pyrimidin-2-yl)-amine |
| 43 | 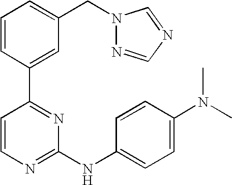 | N,N-Dimethyl-N′-[4-(3-[1,2,4]triazol-1-ylmethyl-phenyl)-pyrimidin-2-yl]-benzene-1,4-diamine |
| 46 | 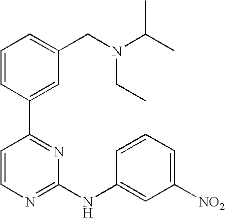 | (4-{3-[(Ethyl-isopropyl-amino)-methyl]-phenyl}-pyrimidin-2-yl)-(3-nitro-phenyl)-amine |
| 50 | 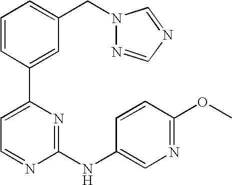 | (6-Methoxy-pyridin-3-yl)-(4-(3-[1,2,4]triazol-1-ylmethyl-phenyl)-pyrimidin-2-yl]-amine |
| 53 | 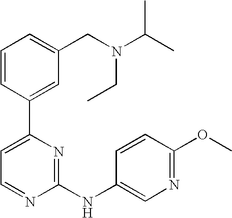 | (4-{3-[(Ethyl-isopropyl-amino)-methyl]-phenyl)-pyrimidin-2-yl)-(6-methoxy-pyridin-3-yl)-amine |
| 54 | 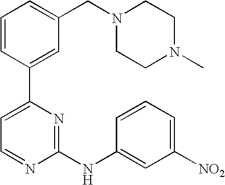 | {4-[3-(4-Methyl-piperizin-1-ylmethyl)-phenyl]-pyrimidin-2-yl}-(3-nitro-phenyl)-amine |
| 55 | 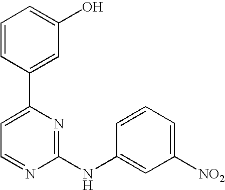 | 3-[2-(3-Nitro-phenylamino)-pyrimidin-4-yl]-phenol |
| 56 | 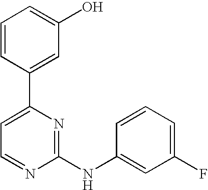 | 3-[2-(3-Fluoro-phenylamino)-pyrimidin-4-yl]-phenol |
| 57 | 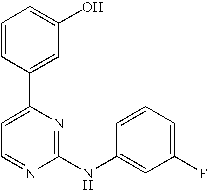 | 3-[2-(3-Fluoro-phenylamino)-pyrimidin-4-yl]-phenol |
| 58 | 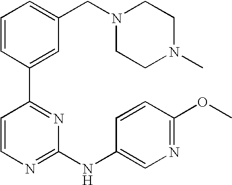 | (6-Methoxy-pyridin-3-yl)-{4-[3-(4-methyl-piperazin-1-ylmethyl)-phenyl]-pyrimidin-2-yl}-amine |
| 59 | 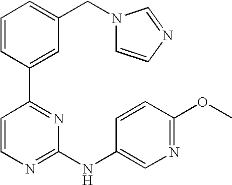 | [4-(3-Imidazol-1-ylmethyl-phenyl)-pyrimidin-2-yl]-(6-methoxy-pyridin-3-yl)-amine |
| 60 | 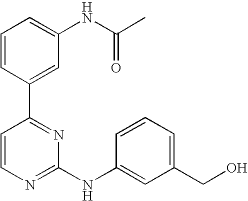 | N-{3-[2-(3-Hydroxymethyl-phenylamino)-pyrimidin-4-yl]-phenyl}-acetamide |
| 67 | 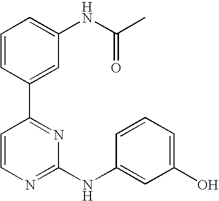 | N-{3-[2-(3-Hydroxy-phenylamino)-pyrimidin-4-yl]-phenyl}-acetamide |
| 69 | 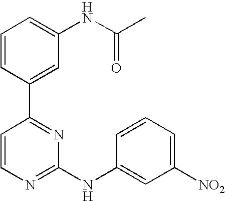 | N-{3-[2-(3-Nitro-phenylamino)-pyrimidin-4-yl]-phenyl}-acetamide |
| 71 | 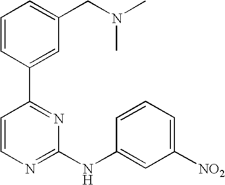 | [4-(3-Dimethylaminomethyl-phenyl)-pyrimidin-2-yl]-(3-nitro-phenyl)-amine |
| 76 | 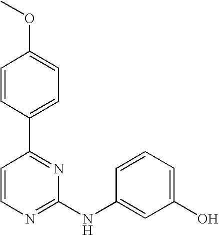 | 3-[4-(4-Methoxy-phenyl)-pyrimidin-2-ylamino]-phenol |
| 79 | 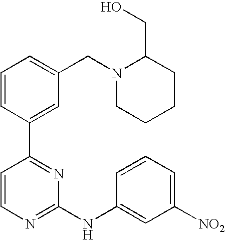 | (1-{3-[2-(3-Nitro-phenylamino)-pyrimidin-4-yl]-benzy}-piperidin-2-yl)-methanol |
| 80 | 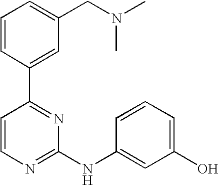 | 3-[4-(3-Dimethylaminomethyl-phenyl)-pyrimidin-2-ylamino]-phenol |
| 81 | 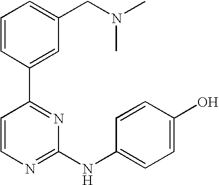 | 4-[4-(3-Dimethylaminomethyl-phenyl)-pyrimidin-2-ylamino]-phenol |
| 82 | 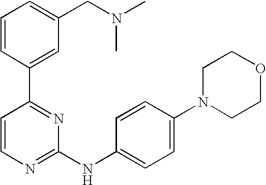 | [4-(3-Dimethylaminomethyl-phenyl)-pyrimidin-2-yl]-(4-morpholin-4-y]-phenyl)-amine |
| 83 | 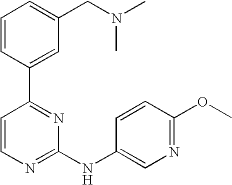 | [4-(3-Dimethylaminomethyl-phenyl)-pyrimidin-2-yl]-(6-methoxy-pyridin-3-yl)-amine |
| 84 | 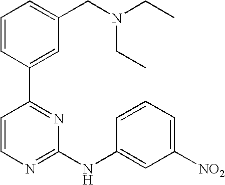 | [4-(3-Diethylaminomethyl-phenyl)-pyrimidin-2-yl]-(3-nitro-phenyl)-amine |
| 85 | 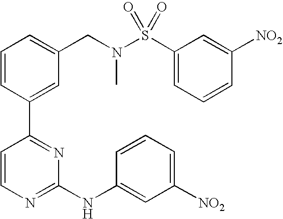 | N-Methyl-3-nitro-N-(3-[2-(3-nitro-phenylamino)-pyrimidin-4-yl]-benzyl}-benzenesulfonamide |
| 86 | 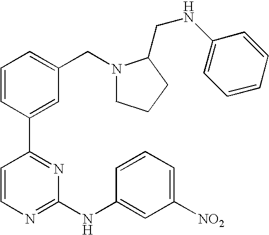 | (3-Nitro-phenyl)-{4-[3-(2-phenylaminomethyl-pyrrolidin-1-ylmethyl)-phenyl}-pyrimidin-2-yl}-amine |
| 87 | 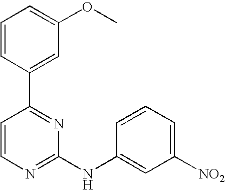 | [4-(3-Methoxy-phenyl)-pyrimidin-2-yl]-(3-nitro-phenyl)-amine |
| 88 | 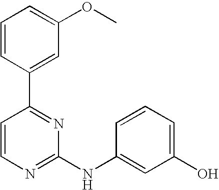 | 3-[4-(3-Methoxy-phenyl)-pyrimidin-2-ylamino]-phenol |
| 91 | 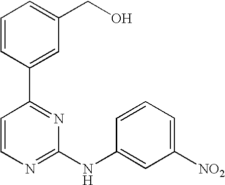 | {3-[2-(3-Nitro-phenylamino)-pyrimidin-4-yl]-phenyl}-methanol |
| 92 | 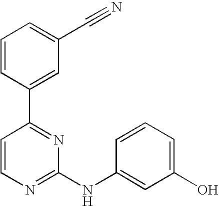 | 3-[2-(3-Hydroxy-phenylamino)-pyrimidin-4-yl]-benzonitrile |
| 94 | 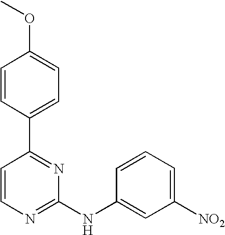 | [4-(4-Methoxy-phenyl)-pyrimidin-2-yl}-(3-nitro-phenyl)-amine |
| 95 | 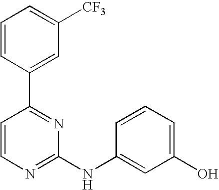 | 3-[4-(3-Trifluoromethyl-phenyl)-pyrimidin-2-ylamino]-phenol |
| 96 | 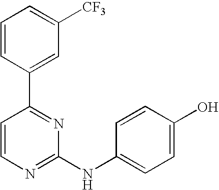 | 4-[4-(3-Trifluoromethyl-phenyl)-pyrimidin-2-ylamino]-phenol |
| 97 | 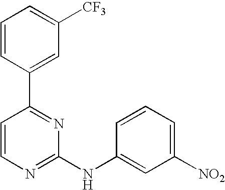 | (3-Nitro-phenyl)-[4-(3-trifluoromethyl-phenyl)-pyrimidin-2-yl]-amine |
| 98 | 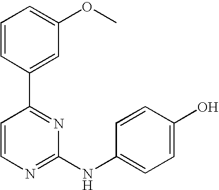 | 4-[4-(3-Methoxy-phenyl)-pyrimidin-2-ylamino]-phenol |
| 99 | 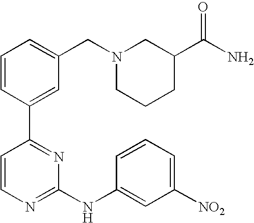 | 1-{3-[2-(3-Nitro-phenylamino)-pyrimidin-4-yl]-benzyl}-piperidine-3-carboxylic acid amide |
| 100 | 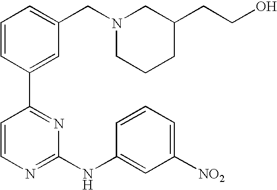 | 2-(1-{3-[2-(3-Nitro-phenylamino)-pyrimidin-4-yl]-benzyl}-piperidin-3-yl)-ethanol |
| 102 | 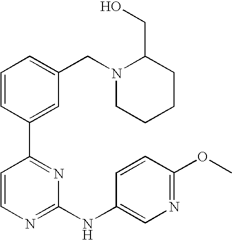 | (1-{3-[2-(6-Methoxy-pyridin-3-ylamino)-pyrimidin-4-yl]-benzyl}-piperidin-2-yl)-methanol |
| 103 | 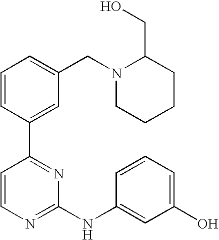 | 3-{4-[3-(2-Hydroxymethyl-piperidin-1-ylmethyl)-phenyl]-pyrimidin-2-ylamino}-phenol |
| 104 | 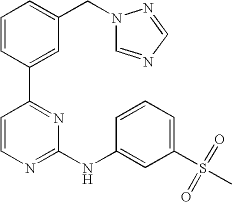 | (3-Methanesulfonyl-phenyl)-[4-(3-[1,2,4]triazol-1-ylmethyl-phenyl)-pyrimidin-2-yl]-amine |
| 105 | 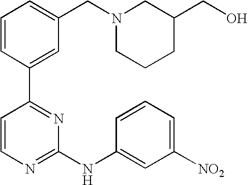 | (1-{3-[2-(3-Nitro-phenylamino)-pyrimidin-4-yl]-benzyl}-piperidin-3-yl)-methanol |
| 106 | 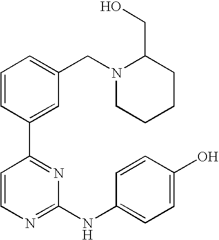 | 4-{4-[3-(2-Hydroxymethyl-piperidin-1-ylmethyl)-phenyl]-pyrimidin-2-ylamino}-phenol |
| 107 | 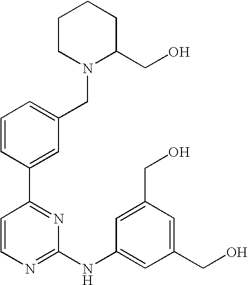 | (1-{3-[2-(3,5-Bis-hydroxymethyl-phenylamino)-pyrimidin-4-yl]-benzyl}-piperidin-2-yl)-methanol |
| 108 | 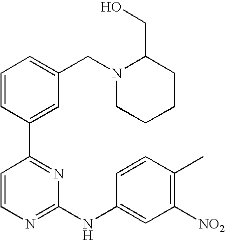 | (1-{3-[2-(4-Methyl-3-nitro-phenylamino)-pyrimidin-4-yl]-benzyl}-piperidin-2-yl)-methanol |
| 109 | 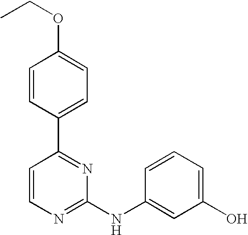 | 3-[4-(4-Ethoxy-phenyl)-pyrimidin-2-ylamino]-phenol |
| 110 | 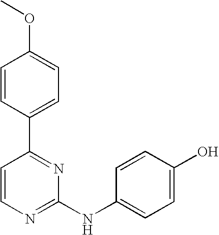 | 4-[4-(4-Methoxy-phenyl)-pyrimidin-2-ylamino]-phenol |
| 112 | 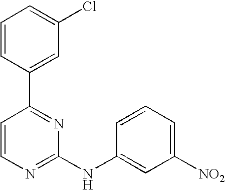 | [4-(3-Chloro-phenyl)-pyrimidin-2-yl]-(3-nitro-phenyl)-amine |
| 113 | 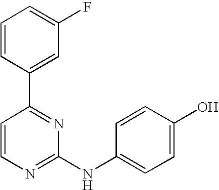 | 4-[4-(3-Fluoro-phenyl)-pyrimidin-2-ylamino]-phenol |
| 114 | 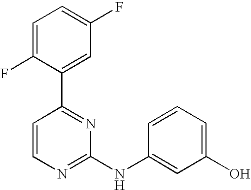 | 3-[4-(2,5-Difluoro-phenyl)-pyrimidin-2-ylamino]-phenol |
| 115 | 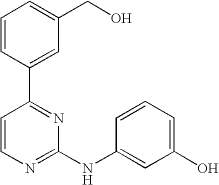 | 3-[4-(3-Hydroxymethyl-phenyl)-pyrimidin-2-ylamino]-phenol |
| 116 | 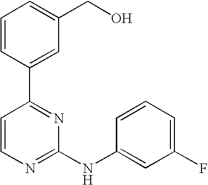 | {3-[2-(3-Fluoro-phenylamino)-pyrimidin-4-yl]-phenyl}-methanol |
| 118 | 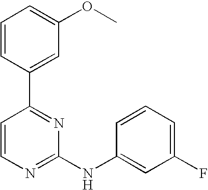 | (3-Fluoro-phenyl)-[4-(3-methoxy-phenyl)-pyrimidin-2-yl]-amine |
| 119 | 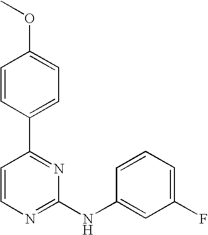 | (3-Fluoro-phenyl)-[4-(4-methoxy-phenyl)-pyrimidin-2-yl]-amine |
| 122 | 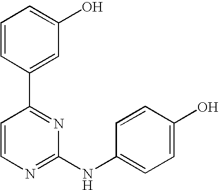 | 3-[2-(4-Hydroxy-phenylamino)-pyrimidin-4-yl]-phenol |
| 123 | 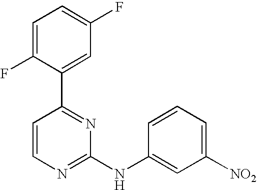 | [4-(2,5-Difluoro-phenyl)-pyrimidin-2-yl]-(3-nitro-phenyl)-amine |
| 124 | 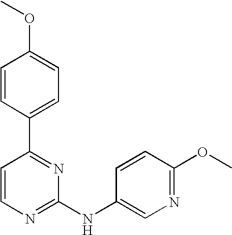 | [4-(4-Methoxy-phenyl)-pyrimidin-2-yl]-(6-methoxy-pyridin-3-yl)-amine |
| 125 | 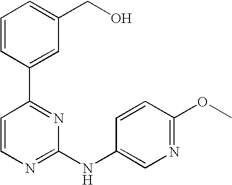 | {3-[2-(6-Methoxy-pyridin-3-ylamino)-pyrimidin-4-yl]-phenyl}-methanol |
| 126 | 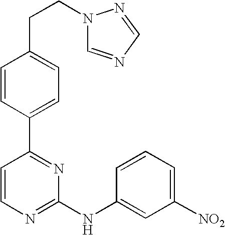 | (3-Nitro-phenyl)-{4-[4-(2-[1,2,4]triazol-1-yl-ethyl)-phenyl]-pyrimidin-2-yl}-amine |
| 127 | 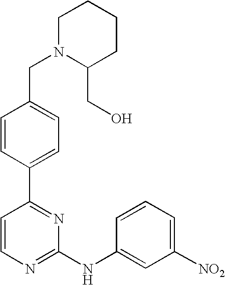 | (1-{4-[2-(3-Nitro-phenylamino)-pyrimidin-4-yl]-benzyl}-piperidin-2-yl)-methanol |
| 129 | 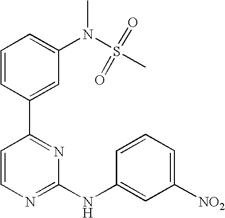 | N-Methyl-N-{3-[2-(3-nitro-phenylamino)-pyrimidin-4-yl]-phenyl]-methanesulfonamide |
| 130 | 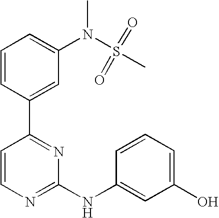 | N-{3-[2-(3-Hydroxy-phenylamino)-pyrimidin-4-yl]-phenyl}-N-methyl-methanesulfonamide |
| 131 | 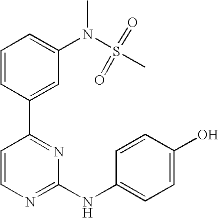 | N-{3-[2-(4-Hydroxy-phenylamino)-pyrimidin-4-yl]-phenyl}-N-methyl-methanesulfonamide |
| 132 | 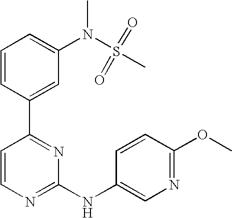 | N-{3-[2-(6-Methoxy-pyridin-3-ylamino)-pyrimidin-4-yl]-phenyl}-N-methyl-methanesulfonamide |

In a particularly preferred embodiment, the cyclin-dependent kinase inhibitor has the following structure:

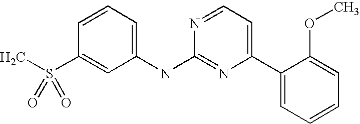

### Compound # 133

The compounds according to any one of general Formula I or Ia have been described in WO2005/012262, which is incorporated herein in its entirety. Methods of manufacturing the compounds according to any one of general formula I or Ia may be found in said application.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al., Protective Groups in Organic Synthesis, Wiley, N.Y. (1991).

Suitable examples of salts of the compounds according to the invention with inorganic or organic acids are hydrochloride, hydrobromide, sulfate, phosphate. Salts, which are unsuitable for pharmaceutical uses, but which can be employed, for example, for the isolation or purification of free compounds or their pharmaceutically acceptable salts, are also included.

All stereoisomers of the compounds of the instant invention are contemplated, either in admixture or in pure or substantially pure form. The definition of the compounds according to the invention embraces all possible stereoisomers and their mixtures. In particular, it embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

It should be understood that solvates (e.g., hydrates) of the compounds of formula I or Ia are also within the scope of the present invention. Methods of solvation are generally known in the art. Accordingly, the compounds of the instant invention may be in the free or hydrate form.

### Therapeutic Use

The compounds according to any one of general Formula I or Ia as provided herein display an unexpected antinociceptive effect.

Thus, in a preferred embodiment, the compounds of any one of general Formula I or Ia may be used in methods and/or pharmaceutical compositions for the treatment of any type of pain, including chronic pain, neuropathic and/or inflammatory pain.

In a particularly preferred embodiment, the compounds of any one of general Formula I or Ia for use in the treatment of any type of pain display an increased selectivity for CDK9 than for other CDKs.

### Pain

Administration of CDK inhibitors according to any one of general Formula I or Ia as disclosed herein to mice suffering from nerve lesion exerts a hypoalgesic effect, in particular in murine models of inflammatory and neuropathic pain.

The discovery that inhibition of a cyclin-dependent kinase is involved in mediating a hypoalgesic effect was unexpected.

Thus, in a preferred embodiment, this invention relates to a method of treating any type of pain comprising administering an effective amount of an inhibitor of cyclin-dependent kinase according to any one of general Formula I or Ia to a subject in need thereof. Specifically, the compounds of any one of general Formula I or Ia may be used for the treatment of chronic, neuropathic and/or inflammatory pain. In a particular preferred embodiment, the compounds of any one of general Formula I or Ia for use in the treatment of any type of pain display an increased selectivity for CDK9 than for other CDKs.

The role of CDK9 in the development of pain is supposed to be based on the following mechanism of action: Both cyclin T1 and CDK9 stimulate the basal promoter activity of TNFα. TNFα is a pro-inflammatory cytokine and pain mediator that controls expression of inflammatory genetic networks. For mediation of cellular TNF receptor responses, the nuclear factor-κB (NFκB) pathway is crucial. TNFα triggers its recruitment to cytokine genes while NfκB interacts with p-TEFb complex for stimulation of gene transcription (Barboric M et al., 2001).

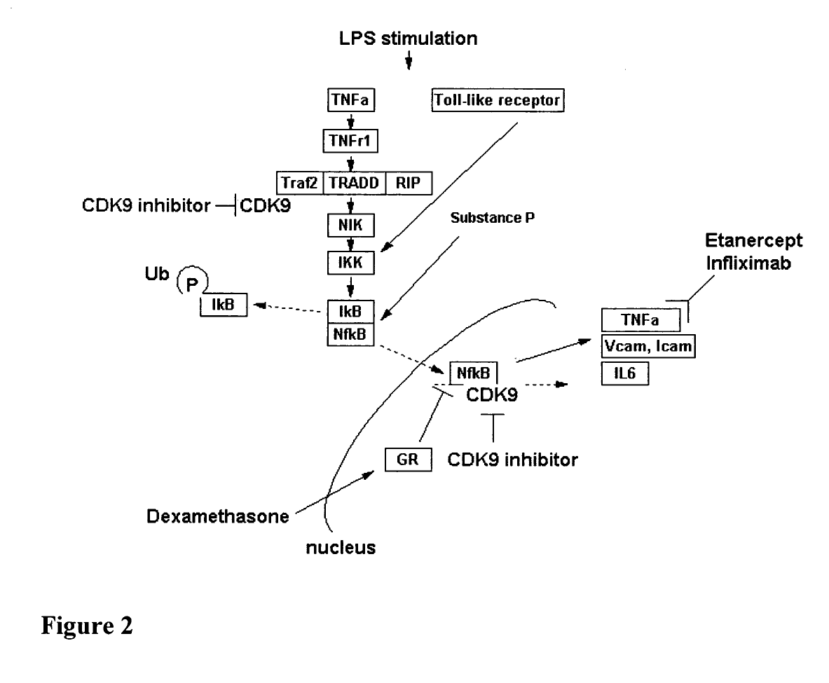

Additionally, it has been shown that CDK9 is a binding partner of TRAF2, a member of the TNFα receptor complex (MacLachlan et al, 1998), while GP130, a subunit of the pro-inflammatory IL6 receptor complex has recently been identified as another potential binding partner of CDK9 (Falco et al, 2002). As a key player in TNFα and interleukin signaling as well as in NfκB mediated expression of several genes (e.g. cytokines as pain mediators), CDK9 can thus be considered as a central target for the treatment of any type of pain, such as inflammatory pain (see FIG. 2).

For the treatment of neuropathic pain, pharmacological action has to take place beyond the blood-brain-barrier (BBB) in the central nervous system (CNS). Microglial cells as the principal immune cells in the CNS, for example, release, upon activation, a variety of noxious factors such as cytokines (TNFα, IL1β, IL6) and other pro-inflammatory molecules (Huwe 2003). Microglia are activated by stimulation of TNFα receptor or Toll-like receptor and signal is mediated via Iκ kinase (IKK) and NfκB leading to transcriptional activation of the cytokines described above. Microglial contribution has been discussed as instrumental in chronic CNS diseases and may contribute to pain perception (Watkins et al, 2003).

Recently it has been shown that the transcription factor NfκB regulates expression of Cyclooxygenase-2 (COX-2) via Interleukin 1β (IL1β) in the spinal cord (Lee et al. 2004). As the major contributor to elevation of spinal prostaglandin E2, the pain mediator COX-2 is already known as a target for a variety of anti-nociceptive/anti-inflammatory drugs. NfκB inhibitors have proven their ability to significantly reduce COX-2 levels and mechanical allodynia as well as thermal hyperalgesia in animal models.

In contrast to COX-2, inhibition of CDK9 action would lead to suppression of a variety of pain mediators instead of just a single one. Thus, anti-nociceptive action of CDK9 inhibitors may be superior compared to e.g. COX-2 inhibitors.

Due to its relevance for NfκB mediated gene transcription, the inhibitory interaction with CDK9 may therefore be a reasonable approach not only for the treatment of acute inflammatory pain, but also for the treatment of chronic pain.

The term “pain” as used herein generally relates to any type of pain and broadly encompasses types of pain such as acute pain, chronic pain, inflammatory and neuropathic pain. In a preferred embodiment of the present invention, “pain” comprises neuropathic pain and associated conditions. The pain may be chronic pain, allodynia (the perception of pain from a normally innocuous stimulus), hyperalgesia (an exaggerated response to any given pain stimulus) and an expansion of the receptive field (i.e. the area that is “painful” when a stimulus is applied), phantom pain or inflammatory pain.

Acute pain types comprise, but are not limited to pain associated with tissue damage, postoperative pain, pain after trauma, pain caused by burns, pain caused by local or systemic infection, visceral pain associated with diseases comprising: pancreatits, intestinal cystitis, dysmenorrhea, Irritable Bowel syndrome, Crohn's disease, ureteral colic and myocardial infarction.

Furthermore, the term “pain” comprises pain associated with CNS disorders comprising: multiple sclerosis, spinal cord injury, traumatic brain injury, Parkinson's disease and stroke. In a preferred embodiment, “pain” relates to chronic pain types comprising headache (for example migraine disorders, episodic and chronic tension-type headache, tension-type like headache, cluster headache, and chronic paroxysmal hemicrania), low back pain, cancer pain, osteoarthritis pain and neuropathic pain, but is not limited thereto.

Inflammatory pain (pain in response to tissue injury and the resulting inflammatory process) as defined herein relates to inflammatory pain associated with diseases comprising connective tissue diseases, rheumatoid arthritis, systemic lupus erythematosus, multiple sclerosis and arthritis, but is not limited thereto.

Neuropathic pain (pain resulting from damage to the peripheral nerves or to the central nervous system itself) includes conditions comprising, but not limited to metabolic neuropathies (e.g., diabetic neuropathy), post-herpetic neuralgia, trigeminal neuralgia, cranial neuralgia, post-stroke neuropathic pain, multiple sclerosis-associated neuropathic pain, HIV/AIDS-associated neuropathic pain, cancer-associated neuropathic pain, carpal tunnel-associated neuropathic pain, spinal cord injury-associated neuropathic pain, complex regional pain syndrome, fibromyalgia-associated neuropathic pain, reflex sympathic dystrophy, phantom limb syndrome or peripheral nerve or spinal cord trauma, nerve transection including surgery, limb amputation and stump pain, pain caused by the side effects of anti-cancer and anti-AIDS therapies, post-surgical neuropathic pain, neuropathy-associated pain such as in idiopathic or post-traumatic neuropathy and mononeuritis, and neuropathic pain caused by connective tissue disease such as rheumatoid arthritis, Wallenberg's syndrome, systemic lupus erythematosus, multiple sclerosis, or polyarteritis nodosa. The neuropathy can be classified as radiculopathy, mononeuropathy, mononeuropathy multiplex, polyneuropathy or plexopathy.

The term “allodynia” denotes pain arising from stimuli which are not normally painful. Allodynic pain may occur other than in the area stimulated.

The term “hyperalgesia” denotes an increased sensitivity to a painful stimulus.

The term “hypoalgesia” denotes a decreased sensitivity to a painful stimulus.

Specifically, the present invention relates to a method for treating the above-referenced types of pain and associated conditions, wherein the term “treating” comprises the prevention, amelioration or treating of pain and associated conditions.

### Pharmaceutical Compositions

Preferred embodiments of the present invention include the administration of compositions comprising at least one cyclin-dependent kinase inhibitor according to any one of general Formula I or Ia as an active ingredient together with at least one pharmaceutically acceptable (i.e. non-toxic) carrier, excipient and/or diluent.

Preferably, the composition comprises at least one cyclin-dependent kinase inhibitor according to any one of general Formula I or Ia as an active ingredient, wherein said at least one cyclin-dependent kinase inhibitor has an increased selectivity for CDK9 than for other CDKs.

Furthermore, the invention also comprises compositions combining at least two inhibitors of CDK and/or pharmaceutically acceptable salts thereof. Said at least two inhibitors may inhibit the same cyclin-dependent kinase or may also inhibit different types of cylin-dependent kinases, e.g. one inhibitor in the composition may inhibit CDK9 while the other inhibitor is capable of inhibiting CDK2, for example.

With regard to pain treatment, an individual pain medication often provides only partially effective pain alleviation because it interferes with just one pain-transducing pathway out of many. Thus, it is also intended to administer CDK inhibitors according to any one of general Formula I or Ia in combination with a pain-reducing (analgesic) agent that acts at a different point in the pain perception process.

An “analgesic agent” comprises a molecule or combination of molecules that causes a reduction in pain perception. An analgesic agent employs a mechanism of action other than inhibition of CDK.

One class of analgesics, such as nonsteroidal anti-inflammatory drugs (NSAIDs), down-regulates the chemical messengers of the stimuli that are detected by the nociceptors and another class of drugs, such as opioids, alters the processing of nociceptive information in the CNS. Other analgesics are local anesthetics, anticonvulsants and antidepressants such as tricyclic antidepressants. Administering one or more classes of drug in addition to CDK inhibitors can provide more effective amelioration of pain.

Preferred NSAIDs for use in the methods and compositions of the present invention are aspirin, acetaminophen, ibuprofen, and indomethacin. Furthermore, cyclooxygenase-2 (COX-2) inhibitors, such as specific COX-2 inhibitors (e.g. celecoxib, COX189, and rofecoxib) may also be used as an analgesic agent in the methods or compositions of the present invention.

Preferred tricyclic antidepressants are selected from the group consisting of Clomipramine, Amoxapine, Nortriptyline, Amitriptyline, Imipramine, Desipramine, Doxepin, Trimipramine, Protriptylin, and Imipramine pamoate.

Furthermore, the use of anticonvulsants (e.g. gabapentin), GABAB agonists (e.g. L-baclofen), opioids, vanniloid receptor antagonists and cannabinoid (CB) receptor agonists, e.g. CB1 receptor agonists as analgesic is also preferred in the methods and compositions in the present invention.

In preparing cyclin-dependent kinase inhibitor compositions of this invention, one can follow the standard recommendations of well-known pharmaceutical sources such as Remington: The Science and Practice of Pharmacy, 19^{th} ed. (Mack Publishing, 1995).

The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, wherein said preparations in addition to typical vehicles and/or diluents contain at least one inhibitor according to the present invention and/or a pharmaceutical acceptable salt thereof as active ingredient.

The pharmaceutical compositions according to the present invention containing at least one inhibitor according to the present invention and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like.

Moreover, suitable binders, lubricants, disintegrating agents and coloring agents may also be incorporated into the tablet or capsule. Powders and tablets may contain about 5 to about 95% by weight of a cyclin-dependent kinase inhibitor according to the Formula I as recited herein or analogues thereof or the respective pharmaceutical active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like.

Suitable disintegrants include starch, methylcellulose, guar gum, and the like.

Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect (s), e.g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g. nitrogen.

For preparing suppositories, a low melting wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified.

Also included are solid form preparations, which are intended to be converted, shortly before use, to liquid form preparations for either oral or parenteral administration. Such liquid forms include solutions, suspensions, and emulsions.

The compounds according to the present invention may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e.g. of methylcellulose, polyvinyl alcohols, or denatured gelatins or starch for holding or containing compositions comprising the active ingredient(s). Capsules with hard shells are typically made of blended or relatively high gel strength gelatins from bones or pork skin. The capsule itself may contain small amounts of dyes, opaquing agents, plasticisers and/or preservatives. Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powders for constitution refer to powder blends containing the active ingredients and suitable diluents which can be suspended e.g. in water or in juice.

Suitable diluents are substances that usually make up the major portion of the composition or dosage form. Suitable diluents include sugars such as lactose, sucrose, mannitol, and sorbitol, starches derived from wheat, corn rice, and potato, and celluloses such as microcrystalline cellulose. The amount of diluent in the composition can range from about 5 to about 95% by weight of the total composition, preferably from about 25 to about 75% by weight, and more preferably from about 30 to about 60% by weight.

The term disintegrants refers to materials added to the composition to support disintegration and release of the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodiumcroscaramellose, alginates such as alginic acid and sodium alginate, clays such as bentonites, and effervescent mixtures. The amount of disintegrant in the composition may range from about 2 to about 20% by weight of the composition, more preferably from about 5 to about 10% by weight.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat corn rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20% by weight of the composition, preferably from about 3 to about 10% by weight, and more preferably from about 3 to about 6% by weight.

Lubricants refer to a class of substances which are added to the dosage form to enable the tablet granules etc. after being compressed to release from the mould or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5% by weight of the composition, preferably from about 0.5 to about 2% by weight, and more preferably from about 0.3 to about 1.5% by weight of the composition.

Glidents are materials that prevent baking of the components of the pharmaceutical composition together and improve the flow characteristics of granulate so that flow is smooth and uniform. Suitable glidents include silicon dioxide and talc.

The amount of glident in the composition may range from about 0.1 to about 5% by weight of the final composition, preferably from about 0.5 to about 2% by weight.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5% by weight of the composition, preferably from about 0.1 to about 1% by weight.

The present invention relates to the administration of compositions containing as active ingredient a cyclin-dependent kinase inhibitor according to any one of general Formula I or Ia to a subject in need thereof for the treatment of any type of pain.

“A subject in need thereof” comprises an animal, preferably a mammal, and most preferably a human, expected to experience any type of pain in the near future or which has ongoing experience of said conditions. For example, such animal or human may have an ongoing condition that is causing pain currently and is likely to continue to cause pain, or the animal or human has been, is or will be enduring a procedure or event that usually has painful consequences. Chronic painful conditions such as diabetic neuropathic hyperalgesia and collagen vascular diseases are examples of the first type; dental work, particularly in an area of inflammation or nerve damage, and toxin exposure (including exposure to chemotherapeutic agents) are examples of the latter type.

In order to achieve the desired therapeutic effect, the respective cyclin-dependent kinase inhibitor has to be administered in a therapeutically effective amount.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated. In the context of the present invention, a therapeutically effective amount comprises, e.g., an amount that reduces pain, in particular inflammatory or neuropathic pain. Specifically, a therapeutically effective amount denotes an amount which exerts a hypoalgesic effect in the subject to be treated.

Such effective amount will vary from subject to subject depending on the subject's normal sensitivity to, e.g., pain, its height, weight, age, and health, the source of the pain, the mode of administering the inhibitor of CDKs, the particular inhibitor administered, and other factors. As a result, it is advisable to empirically determine an effective amount for a particular subject under a particular set of circumstances.

### Salts/Esters

The compounds of Formula I or Ia can be present as salts or esters, in particular pharmaceutically acceptable salts or esters. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

In a preferred embodiment, a pharmaceutically acceptable salt is selected from the group consisting of hydrochloride, dihydrochloride, sulfate, trifluoroacetate, mixture of trifluoroacetate and hydrochloride, tartrate, fumarate, succinate, maleate, citrate, methanesulfonate, bromate and iodate salts.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkane alcohols of 1-12 carbon atoms which may be unsubstituted or substituted, e.g. by a halogen).

### Enantiomers/Tautomers

The present invention also comprises all enantiomers and tautomers of the compounds of Formula I or Ia. The corresponding enantiomers and/or tautomers may be isolated/prepared by methods known in the art.

### Stereo and Geometric Isomers

Some of the compounds of the invention may exist as stereoisomers and/or geometric isomers, e.g. they may possess one or more asymmetric and/or geometric centres and thus, may exist in two or more stereoisomeric and/or geometric forms. The present invention contemplates the use of all the individual stereoisomers and geometric isomers of those inhibitor agents, and mixtures thereof.

### Solvates

The present invention also includes the use of solvate forms of the compounds of the present invention.

### Polymorphs

The invention furthermore relates to the compounds of the present invention in their various crystalline forms, polymorphic forms hydrous forms, and anhydrous forms. It is well established that chemical compounds may be isolated in any of such forms by slightly varying the method of purification and or isolation form the solvents used in the synthetic preparation of such compounds.

### Prodrugs

The invention further includes the compounds of the present invention in prodrug form. Such prodrugs are generally compounds of Formula I or Ia wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo.

Examples of such modifications include ester (for example, any of those described above), wherein the reversion may be carried out be an esterase etc. Other such systems will be well known to those skilled in the art.

The compounds according to any one of general Formula I or Ia can be prepared by any conventional means, known to the skilled artisan. Suitable processes for synthesizing these compounds are provided in WO 2005/012262, which is incorporated herein in entirety.

The invention is further illustrated by the following non-limiting examples.

### EXAMPLES

### Example 1

### I. Behavioral Animal Models for the Analysis of Inflammatory and Neuropathic Pain

Several animal models for the analysis of inflammatory and neuropathic pain are known. Said models share the common feature that after e.g., induction of a nerve lesion (e.g., spared nerve injury, SNI) or after exposing experimental animals to a noxious stimulus (e.g., injection of formalin or carrageenan), the signs of pain as induced by said interventions are measured by quantifiable behavioral components such as, e.g., paw withdrawal threshold to mechanical stimulation with von Frey hairs (or to thermal stimulation using a laser source or licking behaviour). These reactions are interpreted as being equivalent to mechanical and thermal allodynia (hypersensitivity to mechanical stimuli) or hyperalgesia in humans.

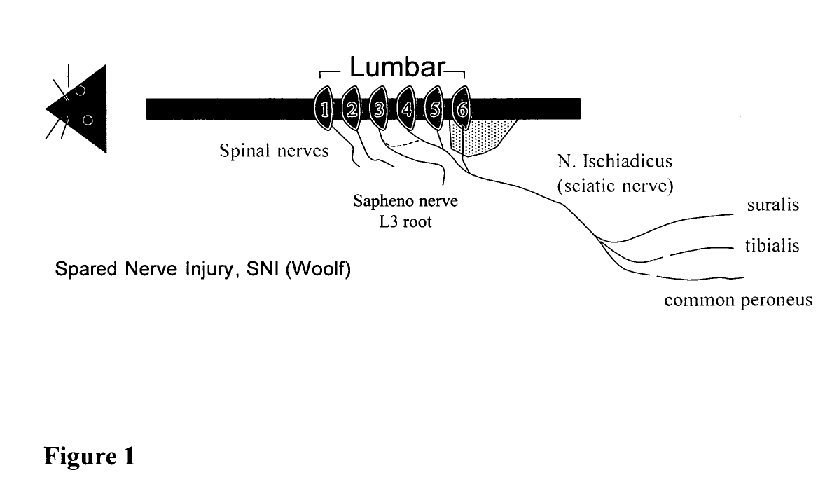

The spared nerve injury model (SNI model, as developed by Decosterd and Woolf (2000), see FIG. 1) is characterized by the induction of clinically relevant nerve lesions and after surgical intervention, subsequent behavioral experiments (e.g., von Frey Assay). Said model constitutes a common nerve injury model which consists of ligation and section of two branches of the sciatic nerve (namely tibial and common peroneal nerves) leaving the sural nerve intact. The SNI model results in early (less than 24 hours), prolonged and substantial changes in mechanical and cold sensitivity that closely mimic the features of clinical neuropathic pain. Animals with these types of nerve injury have been shown to develop abnormal pain sensations and hypersensitivity to mechanical stimuli (allodynia) similar to those reported by neuropathic pain patients.

Alternatively, the formalin assay in mice is a valid and reliable behavioral model of nociception in inflammatory and neuropathic pain. It is sensitive to various classes of analgesic drugs (Hunskaar S, Hole K, Pain. 1987 July; 30(1):103-14.) The noxious stimulus consists of an injection of 10 μl diluted formalin (2% in saline) under the skin of the dorsal surface of the left hindpaw (subcutaneous or interplantar into the left hindpaw). The response is licking and flinching of the injected paw.

For the carrageenan assay a subcutaneous injection of 25 μl of 1% carrageenan (in saline) into a single hind paw (ipsi-lateral paw) of mice is applied. Subsequent inflammation results in long lasting swelling and hypersensitivity (against mechanical and thermal stimuli) of the paw. The carrageenan assay is a standard laboratory assay used to predict anti-inflammatory activity of test compounds. Paw edema measurements and Hargreaves Assay (withdrawal of paws due to thermal stimulation via a light source) are used for read out.

Regarding the present invention, the effect of administration of cyclin-dependent kinase (CDK)-inhibiting compounds according to any one of general Formula I or Ia as disclosed herein on the development of inflammatory and neuropathic pain is assayed in a SNI model, in a carrageenan and in a formalin assay. The experimental procedure and results are described in detail below.

### Example 2

### A. Spared Nerve Injury (SNI)

### Model of Chronic Neuropathic Pain

As outlined above, the spared nerve injury (SNI) model (see FIG. 1) involves a lesion of two of the three terminal branches of the sciatic nerve (tibial and common peroneal nerves) of experimental animals, leaving the sural nerve intact. SNI results in mechanical and thermal allodynia in the non-injured sural nerve skin territory (Decosterd and Woolf, Pain 2000; 87:149-158. (2) Tsujino et al., Mol. Cell. Neurosci. 2000; 15:170-182).

### 1. Induction of Spared Nerve Injury (Nerve Lesion) in Wildtype Mice

Wildtype mice (strain C3HeB/FeJ) (age, sex and weight matched) are anesthetized with Hypnorm (0.315 mg/ml fentanyl citrate+10 mg/ml fluanisone; Janssen)/Hypnovel (5 mg/ml midazolam; Roche Applied Sciences)/water at a ratio of 1:1:2 at 4 μl/g prior to surgical preparation.

Subsequently, an incision is made under aseptic precautions in the ipsi-lateral right hind leg of all mice just above the level of the knee, exposing the three terminal branches of the sciatic nerve: the common peroneal, tibial, and sural nerves. The common peroneal and tibial nerves are ligated tightly with 7/0 silk and sectioned distal to the ligation removing about 2 mm of distal nerve stump. The sural branch remains untouched during the procedure (denoted herein “SNI ipsi”). The overlying muscle and skin is sutured, and the animals are allowed to recover and to permit wound healing. In the same mice the sciatic nerve branches of the contra-lateral left hind leg are exposed but not lesioned (denoted herein “SNI contra-lateral”). Mice that underwent spared nerve injury are hereinafter denoted “SNI mice”.

### 2. Administration of CDK-Inhibiting Compounds to SNI Mice

After recovery from surgery and wound healing, SNI mice receive per oral (p.o.) injections of CDK-inhibiting compounds.

30 mg/kg of a CDK inhibitor, dissolved in 400 μl of 2% Hydroxprolylcellulose; 0.25% Lactic Acid (85% solution) is administered via per oral application 30 min prior to von Frey measurements (mechanical allodynia). As a negative control, the same amount (400 μl) of 2% Hydroxprolylcellulose; 0.25% Lactic Acid (85% solution) vehicle is administered by a single per oral application 30 min prior to von Frey measurements.

Injection of inhibitor or vehicle, and subsequent measurements of paw withdrawal threshold to mechanical stimulation in von Frey assays are performed at day 107 post SNI. Reflex nociceptive responses to mechanical stimulation are measured in a von Frey assay 30 min after each injection.

The effect of administration of CDK inhibitors to SNI mice on the development of mechanical allodynia is analyzed in a von Frey assay, as described below.

### 3. Behavioral Testing of SNI Mice After Administration of CDK-Inhibiting Compounds (Von Frey Assay)

Mice that underwent SNI and subsequent administration of the compounds of the present invention are tested for signs of mechanical allodynia post nerve injury and post administration in a von Frey assay (Decosterd and Woolf, Pain 2000; 87:149-158). This assay determines the mechanical threshold upon which a stimulus, which normally is not painful, is recognized by an animal as uncomfortable or painful. SNI ipsi and SNI contra baselines, respectively, are established.

Mechanical thresholds of SNI mice are quantified using the up-down method based on Chaplan et al. (1994) and Malmberg and Basbaum (1998).

Mice are placed in plexiglass cylinders of about 9.5 cm in diameter, 14 cm high with four vent holes toward the top and a plexiglass lid. The cylinders are placed on an elevated mesh surface (7×7 mm squares). Prior to the day of testing, the mice are acclimated to the testing cylinders for 1-2 hours. On the day of testing the mice are acclimated to the cylinders for about an hour, wherein the acclimation time depends on factors such as the strain of the mouse and the number of times they have been tested previously. In general, testing may begin once the mice are calm and stop exploring the new environment.

For testing mice, filaments 2.44, 2.83, 3.22, 3.61, 3.84, 4.08, and 4.31 (force range=0.04 to 2.0 g) are used. The 3.61 mN filament is applied first. Said filament is gently applied to the plantar surface of one paw, allowed to bend, and held in position for 2-4 seconds. Whenever a positive response to the stimulus (flexion reaction) occurs the next weaker von Frey hair is applied; whenever a negative response (no reaction) occurs the next stronger force is applied. The test is continued until the response to 4 more stimuli after the first change in response has been obtained. The highest force tested is 4.31. The cut-off threshold is 2 g.

The series of scores (i.e, “flexion reaction” and “no reaction”) and the force of the last filament applied are used to determine the mechanical threshold as described in Chaplan et al., Journal of Neuroscience Methods. 53(1):55-63, 1994 Jul. The threshold determined is that to which the animal would be expected to respond to 50% of the time. Mice are sacrificed after von Frey measurements were accomplished.

### 4. Effects of Administration of CDK-Inhibiting Compounds on the Development of Neuropathic Pain

Compounds according to any one of general Formula I or Ia are administered to SNI mice as described above. Von Frey measurements are performed as described above. Administration of any one of the compounds of the present invention leads to a significant increase of van Frey threshold levels indicating reduced sensitivity to mechanimal stimuli (reduced allodynia). In comparison, animals treated with vehicle per os alone are expected to display low thresholds indicating high allodynia.

### Example 3

### Formalin Assay

### Model of Inflammatory Processes/Inflammatory and Chronic Neuropathic Pain

The formalin assay in mice is a valid and reliable behavioral model of nociception and is sensitive to various classes of analgesic drugs (Hunskaar S, Hole K, Pain. 1987 July; 30(1):103-14.) The noxious stimulus is an injection of 10 μl diluted formalin (2% in saline) subcutaneous or intraplantar into the left hind paw. The response is licking and flinching of the injected paw. The response shows two phases, which reflect different parts of the inflammatory process (Abbott et al 1995), an early/acute phase 0-5 min post-injection, and a late/chronic phase 5-30 min post-injection. The following protocol describes one possible way to conduct the experiment:

### 1. Injection of Formalin and Administration of CDK-Inhibiting Compound

Age, sex and weight matched wildtype mice (C3HeB/FeJ) are used in this assay. Prior to formalin injection the animals are randomly subdivided into experimental groups of 10 animals each. Thirty minutes prior to formalin injection, a suitable dose of a CDK inhibitor dissolved in (400 μl) of 2% Hydroxprolylcellulose; 0.25% Lactic Acid (85% solution) can be administered by i.p. injection. Similarly, Iκ Kinase (IKK) inhibitor (30 mg/kg) in (400 μl) of 2% Hydroxprolylcellulose; 0.25% Lactic Acid (85% solution) (positive control), or vehicle alone (400 μl) of 2% Hydroxprolylcellulose; 0.25% Lactic Acid (85% solution) (negative control) can be administered by i.p. injection 30 min before formalin injection.

For formalin injection the mouse is held with a paper towel, in order to avoid disturbance of the injection by movements. The injected hind paw is held between thumb and forefinger and 10 μl of formalin (2%) is injected subcutaneously (s.c.) between the two front tori into the plantar hind paw using a Hamilton syringe. The behavior of the formalin- and inhibitor-treated mice is analyzed as described below.

2. Behavioral Analysis of Mice after Injection of Formalin and Administration of CDK-Inhibiting Compound

The behaviour of the formalin-treated mice, i.e. licking and flinching, is monitored by an automated tracking system (Ethovision 3.0 Color Pro, Noldus, Wageningen, Netherlands) over a defined period of time: measurement is initiated 5 min after formalin injection and terminated 30 min after formalin injection. This time frame covers phase II of formalin-induced nociception (pain), which is hyperalgesia.

Two different fluorescent dyes are used for topically marking the injected hind paw (yellow dye) (Lumogenyellow; BASF Pigment, Cologne, Germany) and the contralateral paw (blue dye) (Lumogenviolet; Kremer Pigmente, Aichstetten, Germany) respectively. To determine licking behaviour, mice are monitored with a CCD camera. After monitoring and recording, the video is analyzed using the EthoVision software (Ethovision 3.0 Color Pro, Noldus, Wageningen, Netherlands) or by manual analysis. Fluorescent dot sizes and fluorescence intensities are measured and reduction of fluorescent dot size through licking and biting is calculated. The overall licking time intensity is automatically calculated by comparison of dot size reduction of treated versus untreated paws.

As another variant of assay read out, the licking behaviour of the individual animals is tracked manually based on video files. Licking times are recorded over 30 minutes after formalin injection and subdivided for three different licking zones (dorsum, plantar, toes). Overall licking times can be calculated for each animal as well as each experimental group and be used as a parameter for determination of compound efficacy.

As a result it is found that mice receiving vehicle treatment prior to formalin injection (negative control) display a prolonged licking time and a significant reduction of fluorescent dot size at the formalin-treated paw.

In contrast, a reduction in licking time and in consequence no significant reduction of fluorescent dot size of the formalin-treated paw can be observed in test compound/formalin-treated mice. The same effect, i.e. a reduction in licking time and a minor change in fluorescent dot size, is observed in control mice treated with Ikappa kinase inhibitor (IKK; for function of IKK see FIG. 2, positive control).

This observation is indicative for reduced inflammatory/chronic inflammatory pain perception in CDK9 inhibitor-treated mice and for a hypoalgesic effect of the tested compound.

### Example 4

### Carrageenan Assay in Mice

### Model of Inflammation and Inflammatory Pain

The model of carrageenan induced paw edema is a standard laboratory assay used to predict anti-inflammatory activity and reduction of inflammation-induced pain perception of respective compounds. The following protocol describes one possible way to conduct the experiment.

The basic measurement constitutes in the measurement of edema and mechanical as well as thermal hypersensitivity in response to irritants, such as carrageenan.

Inflammation and resulting inflammatory pain is induced by subcutaneous injection of 25 μl of 1% carrageenan (in saline) into mice hind paw (ipsi-lateral paw). Each group of 10 mice receives administration of a compound according to any one of general Formula I or Ia, 30 mg/kg body weight, vehicle ((400 μl) of 2% Hydroxprolylcellulose; 0.25% Lactic Acid (85% solution)) and saline (physiol. NaCl) by i.p. injection 30 min prior to carrageenan injection. Contra-lateral paws do not receive carrageenan injection.

### 1. Effects of Administration of a CDK-Inhibiting Compound on Carrageenan-Treated Mice

Paw edema induced by carrageenan injection are detected by increased paw size measured from dorsal to plantar at the metatarsus region of the injected (ipsi-lateral) paws. Sizes of ipsi- and contra-lateral paws serve as surrogate markers for inflammation and are measured at several time points after carrageenan injection: before injection (−1), 2 h (2), 3 h (3) 4 h (4), 5 h (5), 6 h (6), 24 h (24) after injection.

The paw size of all mice may increase, e.g., by 2 to 3 mm (+10%) within the first hour after carrageenan injection, independent of the type of treatment substance injected 30 minutes prior to carrageenan. During the time course, mice which received treatment with a CDK-inhibiting compound prior to carrageenan injection may display a reduction of the edema until 24 h after carrageenan injection: the increase in paw size could drop e.g. from 10% down to 8%. In contrast, the paw size of the control mice could increase by 30% in average at this time point. After 24 h post carrageenan injection, the size of all paws treated with carrageenan may increase to reach its maximum at 96 h after injection.

As a read-out of the carrageenan assay, a Hargreaves Assay may be performed, wherein said assay allows the measuring of thermal sensitivity to radiant heat. The Hargreaves assay (Hargreaves et al., 1988) measures nociceptive sensitivity in a freely moving animal by focusing a radiant heat source on the plantar surface of an animal's hindpaw as it stands in a plexiglass chamber. Specifically, the lower side of a paw is exposed to a luminous source, generating a temperature of, e.g. 55° C. Thermal sensitivity is measured as latency between start of exposure and lifting/pulling the exposed paw.

Mice treated with a CDK9 inhibitor as disclosed herein and carrageenan, or with Naproxen and carrageenan, or with solvent and carrageenan, respectively, are subjected to a Hargreaves assay. Mice treated with a CDK inhibitor and carrageenan could display a longer latency, compared to negative control mice. This observation would be indicative for a hypoalgesic effect of the CDK inhibitors as disclosed herein.

### Example 5

### Carrageenan Assay in Rats

### Model of Inflammation and Inflammatory Pain

The following depicts one possible way of performing the carrageenan assay in rats.

Said assay detects analgesic/anti-inflammatory activity in rats with inflammatory pain, following the protocol as described by Winter et al (Proc. Soc. Exp. Biol. Med., 111, 544-547, 1962).

Rats (200-250 g) are injected with a suspension of carrageenan into the lower surface of the right hindpaw (0.75 mg per paw in 0.05 ml physiological saline). Two hours later rats are submitted consecutively to tactile and thermal stimulation of both hindpaws.

For tactile stimulation, the animal is placed under an inverted acrylic plastic box (18×11.5×13 cm) on a grid floor. The tip of an electronic Von Frey probe (Bioseb, Model 1610) is then applied with increasing force first to the non-inflamed and then the inflamed hindpaw and the force required to induce paw-withdrawal is automatically recorded. This procedure is carried out 3 times and the mean force per paw is calculated.

For thermal stimulation, the apparatus (Ugo Basile, Reference: 7371) consists of individual acrylic plastic boxes (17×11×13 cm) placed upon an elevated glass floor. A rat is placed in the box and left free to habituate for 10 minutes. A mobile infrared radiant source (96±10 mW/cm²) is then focused first under the non-inflamed and then the inflamed hindpaw and the paw-withdrawal latency is automatically recorded. In order to prevent tissue damage the heat source is automatically turned off after 45 seconds.

After the behavioral measures, the paw edema is evaluated by measuring the volume of each hindpaw using a digital plethysmometer (Letica, Model 7500), which indicates water displacement (in ml) induced by paw immersion.

10 rats are studied per group. The test is performed blind.

The test substance, such as a CDK inhibitor according to any one of general Formula I or Ia as presented herein, will be evaluated at 2 doses (10 and 30 mg/kg), administered p.o. 60 minutes before the test, and compared with a vehicle control group.

Morphine (128 mg/kg p.o.) and acetylsalicylic acid (512 mg/kg p.o.), administered under the same experimental conditions, will be used as reference substances.

The experiment will therefore include 6 groups. Data will be analyzed by comparing treated groups with vehicle control using unpaired Student's t tests.

Rats treated with a CDK9 inhibitor as disclosed herein and carrageenan, or with Naproxen and carrageenan, or with solvent and carrageenan, respectively, are subjected to a Hargreaves assay. Rats treated with a CDK inhibitor and carrageenan should display a longer latency, compared to negative control rats. This observation would be indicative for a hypoalgesic effect of the CDK inhibitors as disclosed herein.

### Example 6

### A. LPS In Vivo Assay (LPS)

### Model of Cytokine Repression In Vivo

For the LPS induced model of septic shock, mice receive an intraperitoneal (i.p.) injection of 30 μg bacterial Lipopolysaccharide (LPS; L2630 SIGMA) in saline. Said LPS-mediated initiation of the inflammatory signalling cascade results in increasing blood serum concentrations of cytokines such as e.g. TNFα, IL-6 and IL1β. Blood can be taken from these animals at defined time points. Thereafter, serum will be separated and the samples can be stored at −80° C. until cytokine concentrations are measured using commercial ELISA assays. (A L Moreira et al., Braz J Med Biol Res 1997; 30:1199-1207).

It has been recognized that inflammatory mediators such as the cytokines TNFα, IL6 and IL1β can contribute to persistent pain states as well as inflammatory disorders. After being released from immune cells like macrophages in peripheral and microglia in CNS tissues, these mediators seem to play a pivotal role not only in inflammatory and neuropathic pain but also in inflammatory disorders such as rheumatoid arthritis (F Marchand et al., Nat Rev Neurosci 2005; 6 (7); 521-532). Thus, inhibition of tumor necrosis factor α (TNFα) represents a relevant target for the treatment of inflammatory diseases as well [Lavagno et al., Eur J Pharmacol 2004; 501, 199-208].

The LPS in vivo assay can be used as a powerful model to address repression of cytokine expression by pharmacological treatments.

### 1. Induction of Cytokine Expression in Wildtype Mice

Wildtype mice (strain C3HeB/FeJ) (age, sex and weight matched) were injected with 30 μg LPS (SIGMA) intraperitoneally. 90 minutes after LPS administration, these animals were anaesthetized with 0.1 ml/10 g bodyweight Ketamine-Rompun (50/20 mg/ml) and blood for serum preparation was taken via cardiac puncture.

### 2. Administration of CDK-Inhibiting Compounds to LPS Mice

Pharmacological treatment groups (n=4) of LPS mice received per os (p.o.) injections of CDK-inhibiting compounds or the vehicle (negative control), respectively.

10 or 30 mg/kg (compound per bodyweight) of a CDK inhibitor, dissolved in 1% carboxy-methylcellulose (SIGMA) was administered as a single p.o. dosage 30 min prior to LPS stimulation. Vehicle control was administered in the same manner.

90 minutes (min) after LPS stimulation, blood samples were taken from the mice. Previously, the 90 min time point had been identified as the peak of TNF alpha expression in this animal model by a time course experiment.

The effect of pharmacological treatment with CDK inhibitors on cytokine levels in LPS mice was analyzed in commercial ELISA assays as described below.

3. Determination of Cytokine Blood Serum Concentrations in LPS Mice after Administration of CDK-Inhibiting Compounds

Blood samples (˜500 μl/animal) from the LPS animals were incubated on wet ice for 30 min after cardiac puncture. Afterwards the samples were centrifuged for 15 min at 13,000 rpm. Serum was separated from the clot and stored frozen at −80° C.

Serum concentrations of TNF alpha and IL6 within the samples were measured by using commercial ELISA Kits (Natutec) according to the manufacturers instructions.

### 4. Results

30 mg compound/kg body weight formulated in 200 μl 10% DMSO, 5% Tween 80, 4% Tris 1 M pH8, 81% PBS were administered as a single p.o. dosage 30 min prior to LPS stimulation. A vehicle control with 200 μl 10% DMSO, 5% Tween 80, 4% Tris 1M pH8, 81% PBS was administered in the same manner. The determination of blood serum concentration in LPS mice after administration of CDK-inhibiting compounds was performed as described above.

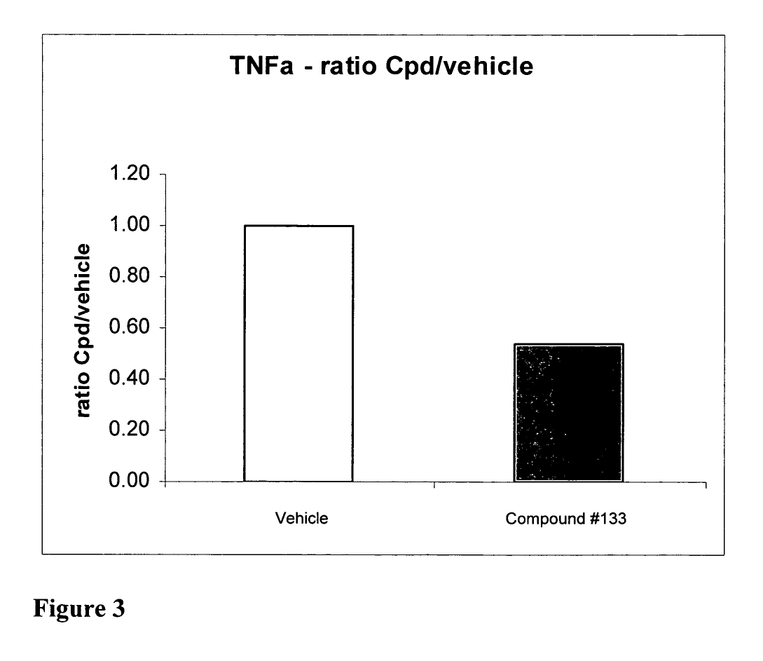

The results are shown in FIG. 3.

FIG. 3 shows reduction of LPS-induced cytokine levels by compound #133 as exemplified for TNFα-levels. Compound #133 reduces the signalα signal by 50% compared to the DMSO (vehicle control). These finding indicates that CDK-inhibitory compound # 133 is an effective suppressor of expression of LPS-induced cytokines.

### Example 7

### A. In Vitro THP-1 Assay

### In Vitro Model of Cytokine Inhibition

The human THP-1 cell line can be utilized as an in vitro model of cytokine expression as mediated by Lipopolysaccharide (LPS) or Tumor Necrosis Factorα [TNFα].

Monocytic THP-1 cells (ATCC; TIB-202) can be differentiated into macrophage-like cells expressing pro-inflammatory cytokines like TNFα, IL6 and IL10 upon induction with LPS or by TNFα (autocrine induction) itself.

It has been recognized that inflammatory mediators such as the cytokines TNFα, IL6 and IL1β can contribute to persistent pain states as well as to inflammatory disorders. After being released from immune cells like macrophages in peripheral and microglia in CNS tissues, these mediators seem to play a pivotal role not only in inflammatory and neuropathic pain but also in inflammatory disorders such as rheumatoid arthritis (F Marchand et al., Nat Rev Neurosci 2005; 6 (7); 521-532). Hence inhibition of tumor necrosis factor α (TNFα) represents a relevant target in the treatment of inflammatory disorders as well [Lavagno et al., Eur J Pharmacol 2004; 501, 199-208].

Therefore, the THP-1 in vitro assay can be used as a powerful screening model to address pharmacological inhibition of cytokine expression (U Singh et al, Clin Chem 2005; 51 (12); 2252-6], K Rutault et al., J Biol Chem 2001; 276 (9); 6666-74].

### 1. Growth and Differentiation of THP-1 Cells

THP-1 cells are grown in modified RPMI-1640 medium (ATCC, Cat. No. 30-2001) supplemented with 10% FCS and 1% Pen/Strep. For cytokine inhibition assays, cells are seeded at a density of 5×10⁵ cells/ml into 6-well plates in standard growth medium supplemented with 100 ng/ml PMA (Sigma, P1585) to induce differentiation into macrophage-like cells. After 24 hours, the medium is replaced with standard growth medium (without PMA) and the cells are incubated for another 48 hours to complete differentiation.

2. Treatment of Differentiated THP-1 Cells with CDK-Inhibiting Compounds and LPS Stimulation

After 72 h of differentiation, the medium is replaced with serum free growth medium, and CDK-inhibiting compounds as well as reference compounds such as positive and negative controls, each dissolved in DMSO are added at concentrations ranging from 0.5 to 5 μM (final concentration of DMSO in the well is 0.1%). Cells are incubated for 60 min with compounds prior to stimulation with 100 ng/ml LPS (Sigma, L2630) for another 4-48 hours. Supernatants are collected and assayed immediately for cytokine expression, e.g. for TNFα, IL-6 and IL-1β using commercially available sandwich ELISA assays (eBioscience, Cat. No 88-7346, 88-7066, 88-7010) or kept frozen at 20° C. until evaluation.

### 3. Determination of Cytokine Concentrations in THP-1 Supernatant After Administration of CDK-Inhibiting Compounds

Concentrations of TNFα, IL6 and IL1β within the cell culture supernatants are measured by using commercial ELISA Kits (eBioscience) according to the manufacturer's instructions.

4. Effects of Treatment with CDK-Inhibiting Compounds on the Protein Expression of Cytokines in THP-1 Cell Supernatants

Compound #133 was administered to differentiated THP-1 cells in triplicates as described above (see section 2.). After 60 min of pre-incubation with test or reference compound (a p38 inhibitor and an IKK-inhibitor, respectively) alone, cells were stimulated with LPS. After incubation for 4-48 h, supernatants were collected and ELISA based determinations of cytokine supernatant concentrations were performed as described in section 3, supra.

Comparison of cells treated with compound # 133 versus cells treated with vehicle (DMSO) displayed a significant inhibitory effect of compound #133 on TNFα and IL6 protein concentration in the cell supernatant. Compared to the reference compounds, compound #133 exhibited a similar or better inhibition of TNFα/IL6 expression.

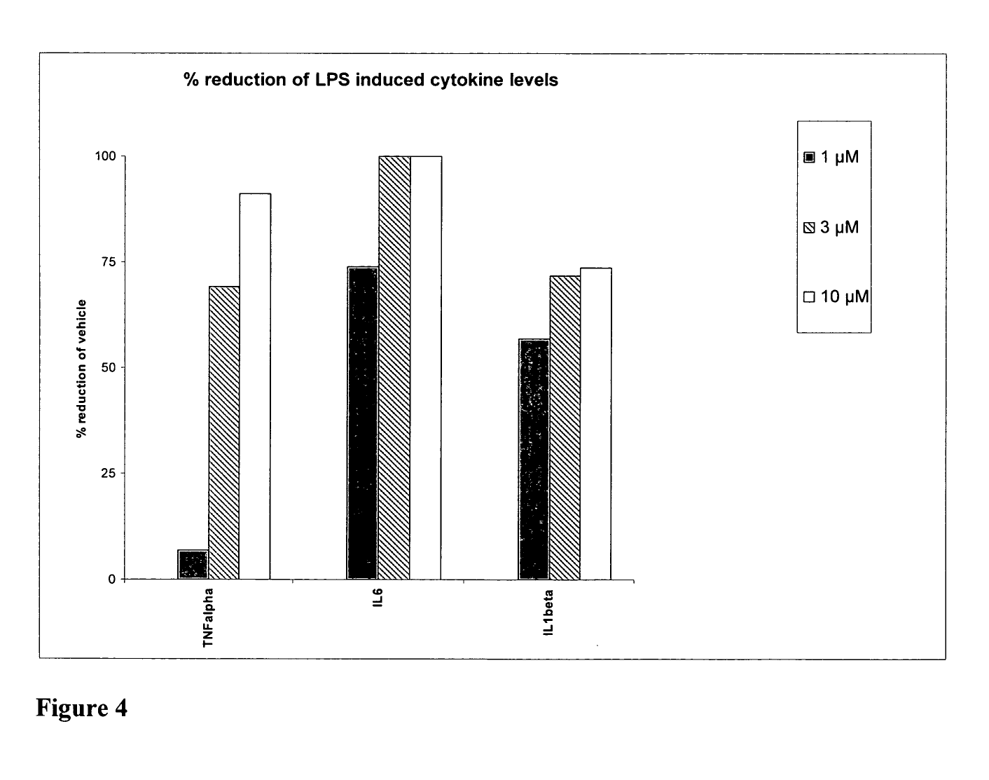

The effects of administration of compound # 133 on expression of TNFα in LPS induced THP-1 macrophages are shown in FIG. 4.

FIG. 4 depicts the reduction of LPS-induced cytokine levels of up to 100% by administration of compound # 133 as exemplified for TNFα-, IL6-, and IL1β-levels. At each concentration, compound # 133 significantly reduced the signalα signal compared to the DMSO (vehicle control). These findings indicate that CDK-inhibitory compound # 133 is an effective suppressor of expression of LPS-induced cytokines, such as TNFα and IL-6.

### Example 8

### A. In Vitro Human Whole Blood Assay

### In Vitro Model of Cytokine Inhibition

The aim of this in vitro assay is to profile anti-inflammatory compounds for their ability to inhibit Lipopolysaccharide (LPS) or Staphylococcus Enterotoxin B (SEB) induced expression of cytokines (e.g. TNFα, IL-1β, IFNγ, IL-2, IL-4, IL-6, IL-8, IL-10, IL-13) in human whole blood. For this purpose, human heparinised whole blood from healthy donors was diluted 1:5 with medium (RPMI 1640, e.g. ATCC #30-2001, supplemented with 4.5 g/L glucose, 4 mM L-glutamine, pyruvate, 1250 U sodium-heparin and 1% Penicillin/Streptomycin), incubated with the anti-inflammatory compound or solvent for 30-60 minutes and then stimulated with 20 ng/ml to 1 μg/ml LPS or SEB for 3 to 48 hours. Supernatants were harvested and analysed for cytokine content, e.g. by ELISA.

### 1. Results.

In the human whole blood assay, TNF α levels were reduced by compound #133 by 94% at a concentration of 30 μM, 85% at a concentration of 10 μM, 60% at a concentration of 3 μM, and 15% at a concentration of 0.3 μM. From this data an IC50 value below 3 μM can be deduced. These findings indicate that CDK-inhibitory compound # 133 is an effective suppressor of expression of Lipopolysaccharide (LPS) or Staphylococcus Enterotoxin B (SEB) induced cytokines.

### Example 9

### A. In Vitro Kinase Inhibition Assays

IC50 profiles of the compounds according to general Formula I or Ia of the present invention can be determined for cyclin-dependent kinases CDK2/CycA, CDK4/CycD1, CDK6/CycD1 and CDK9/CycT in enzymatic kinase inhibition assays in vitro. IC50 values as obtained in these assays are used for evaluating the specific selectivity and potency of the compounds with respect to CDK9 inhibition.

Results obtained in these assays are used to select compounds displaying specificity for CDK9. Specifically, it is intended to distinguish the CDK9-specific compounds from other compounds having significant inhibitory potency also with regard to other CDKs, i.e. on some or all of CDKs 2, 4 and 6. This separation is essential in order to avoid adverse (cytostatic/cytotoxic) effects, which may occur upon inhibition of cell cycle relevant CDKs 2, 4 and 6.

Furthermore, these data are used to establish structure activity relationships (SAR) supporting the design of new and even improved structures/compounds with respect to potency and selectivity.

### 1. Test Compounds

Compounds are used as 1×10⁻² M stock solutions in 100% DMSO, 100 μl each in column 2 of three 96-well V-shaped microtiterplates (in the following, said plates are referred to as “master plates”).

Subsequently, the 1×10⁻² M stock solutions in column 2 of the master plates are subjected to a serial, semi-logarithmic dilution using 100% DMSO as a solvent, resulting in 10 different concentrations, the dilution endpoint being 3×10⁻⁷ M/100% DMSO in column 12. Column 1 and 7 are filled with 100% DMSO as controls. Subsequently, 2×5 μl of each well of the serial diluted copy plates are aliquoted in 2 identical sets of “compound dilution plates”, using a 96-channel pipettor.

On the day of the kinase inhibition assay, 45 μl H₂O are added to each well of a set of compound dilution plates. To minimize precipitation, H₂O is added to the plates only a few minutes before the transfer of the compound solutions into the assay plates. The plates are shaken thoroughly, resulting in “compound dilution plates/10% DMSO” with a concentration of 1×10⁻³ M/10% DMSO to 3×10⁻⁸ M/10% DMSO in semilog steps. These plates are used for the transfer of 5 μl compound solution into the “assay plates”. The compound dilution plates are discarded at the end of the working day. For the assays (see below), 5 μl solution from each well of the compound dilution plates are transferred into the assay plates. The final volume of the assay is 50 μl. All compounds are tested at 10 final assay concentrations in the range from 1×10⁻⁴ M to 3×10⁻⁹ M. The final DMSO concentration in the reaction mixtures is 1% in all cases.

### 2. Recombinant Protein Kinases

For the determination of inhibitory profiles, the following 4 protein kinases are used: CDK2/CycA, CDK4/CycD1, CDK6/CycD1 and CDK9/CycT. Said protein kinases are expressed in Sf9 insect cells as human recombinant GST-fusion proteins or His-tagged proteins by means of the baculovirus expression system. Kinases are purified by affinity chromatography using either GSH-agarose (Sigma) or Ni-NTH-agarose (Qiagen). The purity of each kinase is determined by SDS-PAGE/silver staining and the identity of each kinase is verified by western blot analysis with kinase specific antibodies or by mass spectroscopy.

### 3. Protein Kinase Assay

All kinase assays are performed in 96-well FlashPlates™ from Perkin Elmer/NEN (Boston, Mass., USA) in a 50 μl reaction volume. The reaction mixture is pipetted in four steps in the following order:
20 μl of assay buffer (standard buffer)
5 μl of ATP solution (in H₂O)
5 μl of test compound (in 10% DMSO)
10 μl of substrate/10 μl of enzyme solution (premixed)

The assay for all enzymes contains 60 mM HEPES-NaOH, pH 7.5, 3 mM MgCl₂, 3 mM MnCl₂, 3 μM Na-orthovanadate, 1.2 mM DTT, 50 μg/ml PEG20000, 1 μM [γ-³³P]-ATP (approx. 5×1005 cpm per well).

The following amounts of enzyme and substrate are used per well:

| | | Kinase | Kinase | | Substrate |
|---|---|---|---|---|---|
| # | Kinase | Lot # | ng/50 μl | Substrate | ng/50 μl |
| 1 | CDK2/CycA | SP005 | 100 | Histone H1 | 250 |
| 2 | CDK4/CycD1 | SP005 | 50 | Rb—CTF (Lot 009) | 500 |
| 3 | CDK6/CycD1 | SP003 | 400 | Rb—CTF (Lot 009) | 500 |
| 4 | CDK9/CycT | 003 | 100 | Rb—CTF (Lot 009) | 1000 |

Reaction mixtures are incubated at 30° C. for 80 minutes. The reaction is stopped with 50 μl of 2% (v/v) H₃PO₄, plates are aspirated and washed two times with 200 μl H₂O or 200 μl 0.9% (w/v) NaCl. Incorporation of ³³P is determined with a microplate scintillation counter (Microbeta, Wallac).

All assays are performed with a BeckmanCoulter/Sagian robotic system.

### 4. Evaluation of Raw Data

The median value of the counts in column 1 (n=8) of each assay plate is defined as “low control”. This value reflects unspecific binding of radioactivity to the plate in the absence of a protein kinase but in the presence of the substrate. The median value of the counts in column 7 of each assay plate (n=8) is taken as the “high control”, i.e. full activity in the absence of any inhibitor. The difference between high and low control ss referred to as 100% activity. As part of the data evaluation, the low control value from a particular plate ss subtracted from the high control value as well as from all 80 “compound values” of the corresponding plate. The residual activity (in %) for each well of a particular plate is calculated by using the following formula:

Res. Activity (%)=100×[(cpm of compound−low control)/(high control−low control)]

The residual activities for each concentration and the compound IC50 values are calculated using Quattro Workflow V2.0.1.3 (Quattro Research GmbH, Munich, Germany; world wide web url: www(dot)quattro-research(dot)com). The model used is “Sigmoidal response (variable slope)” with parameters “top” fixed at 100% and “bottom” at 0%.

### 5. Results

The IC50 value on CDK9/CycT for compound #133 was 37.6 nM.

### REFERENCES

Barboric M. et al., NfκB Binds P-TEFb to Stimulate Transcriptional Elongation by RNA Polymerase II. Molecular Cell, 2001, Vol. 8, 327-337
Besson J. M., The neurobiology of pain. Lancet, 1999, 353(9164), 1610-1615
Brower, New paths to pain relief. Nat Biotechnol, 2000, 18(4), 387-391
Chao S. H. and Price D. H., Flavopiridol inactivates P-TEFb and blocks most RNA polymerase II transcription in vivo. J Biol Chem, 2001, 276(34), 31793-9
Chaplan S R, Bach F W, Pogrel J W, Chung J M, and Yaksh, T L. (1994) Quantitative assessment of tactile allodynia in the rat paw. J Neurosci Methods 53: 55-63.
Dai Y. and Grant S., Cyclin-dependent kinase inhibitors. Curr Opin Pharmacol, 2003, 3(4), 362-370
Falco G. D. et al., CDK9, a member of the cdc2-like family of kinases, binds to gp130, the receptor of the IL-6 family of cytokines. Oncogene, 2002, 21(49), 7464-7470
Feldmann and Maini, Nat Med, 2003, 9 (10); 356-61
Firestein, 2003, Nature 423, 356-361
Han et al.; 2003, Autoimmunity, 28, 197-208
Hargreaves, K: Pain 32(1) (1988 January) 77-88
Huwe et al., Small molecules as inhibitors of cyclin-dependent kinases. Angew Chem Int Ed Engl, 2003, 42(19), 2122-2138
Kim et al., Phosphorylation of the RNA polymerase II carboxyl-terminal domain by CDK9 is directly responsible for human immunodeficiency virus type 1 Tat-activated transcriptional elongation. Mol Cell Biol, 2002, 22(13), 4622-4637.
Koltzenburg M, Neural mechanisms of cutaneous nociceptive pain. Clin J Pain, 2000, 16(3 Suppl), 131-138
Laufer S., Gay S. And Brune K., Inflammation and Rheumatic Diseases—The molecular basis of novel therapies, Thieme, 2003
Lee K. M. et al., Spinal NfκB activation induces COX-2 upregulation and contributes to inflammatory pain hypersensitivity. European Journal of Neuroscience, 2004, Vol. 19, 3375-3381
Liu H. and Herrmann C., Differential Localization and Expression of the CDK9 42k and 55k Isoforms. J Cell Physiol, 2004, 203, 251-260
MacLachlan T. K. et al., Binding of CDK9 to TRAF2. J Cell Biochem, 1998, 71(4), 467-478
Malmberg A B and Basbaum A I. (1998) Partial sciatic nerve injury in the mouse as a model of neuropathic pain: behavioral and neuroanatomical correlates. Pain 76: 215-2
Meijer L, Leclerc S., Leost M., Properties and potential applications of chemical inhibitors of cyclin-dependent kinases, Pharmacol Ther 1999, 82(2-3):279-284
Sausville, E. A. Trends Molec. Med. 2002, 8, S32-S37
Tian B. et al., Identification of direct genomic targets downstream of the NfκB transcription factor mediating TNF signaling. JBC, 2005, as manuscript M500437200
Wang D, et al., Inhibition of human immunodeficiency virus type 1 transcription by chemical cyclin-dependent kinase inhibitors. J. Virol. 2001; 75: 7266-7279
Watkins L. R. et al., Glial proinflammatory cytokines mediate exaggerated pain states: implications for clinical pain. Adv Exp Med. Biol., 2003, 521, 1-21
West et al.; 2001, Journal of Virology 75(18), 8524-8537
Zhou M. et al., Coordination of transcription factor phosphorylation and histone methylation by the P-TEFb Kinase during human immunodeficiency virus type I transcription, J. Virol 2004, 78(24):13522-13533

What is claimed is:

1. A method for the treatment of pain comprising administering a therapeutically effective amount of at least one inhibitor of a cyclin-dependent kinase (CDK) to a subject in need thereof.

2. The method of claim 1, wherein said at least one inhibitor comprises at least one compound selected from the group consisting of a [4-(3-substituted-phenyl)-pyrimidin-2-yl]-phenyl-amine and a [4-(3-substituted-phenyl)-pyrimidin-2-yl]-(pyridine-3-yl)-amine.

3. The method of claim 1, wherein said at least one inhibitor of a cyclin-dependent kinase is characterized by general Formula I, 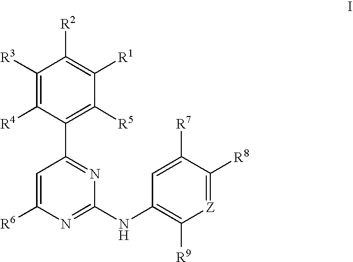
wherein:
Z is CR¹⁰ or N;
one of R¹ and R² is selected from (CH₂)ₘR¹¹, (CH₂)ₘR¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)nR¹¹, (CH₂)ₘCONR¹²R¹³, (CH₂)ₘCO(CH₂)ₙR¹¹ and (CH₂)COR¹²; where m is 0, 1, 2, 3 or 4, and n is 1, 2, 3 or 4;
the other of R¹ and R² is H or R¹¹;
R³ and R⁵ are both H;
R⁴ is H or R¹¹;
R⁶ is H or (CH₂)ₚR¹¹, where p is 0 or 1;
R⁷, R⁹ and R¹⁰ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;
each R¹¹ is independently halogen, NO₂, CN, (CH₂)_{q}OR¹³, (CH₂)ᵣNR¹³R¹⁴, NHCOR¹³, CF₃, COR¹³, R¹³, CONR¹³R¹⁴, SO₂NR¹³R¹⁴, SO₂R¹³, OR¹², NR¹³SO₂R¹⁴, OCH₂CH₂OH, OCH₂CH₂OMe, NR¹³SO₂R¹³, (CH₂)ₛNR¹²R¹³, morpholine, piperidine or piperazine, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
provided that:
when -Z is CR¹⁰ and R⁹ is H, then at least one of R⁷, R⁸ and R¹⁰ is other than OMe; and
when -Z is CR¹⁰ and R⁷, R⁸, and R⁹ are all H, then R¹⁰ is other than OCF₂CHF₂;
and the N-oxide derivatives, prodrug derivatives, protected derivatives, individual isomers, mixtures of isomers, enantiomers, diastereomers thereof, and the pharmaceutically acceptable salts and solvates of such compounds.

4. The method according to claim 1, wherein said at least one inhibitor of a cyclin-dependent kinase is selected from the group consisting of:
4-[4-(3-Nitro-phenyl)-pyrimidin-2-ylamino]-phenol [1];
(4-Nitro-phenyl)-[4-(3-nitro-phenyl)-pyrimidin-2-yl]-amine [2];
[4-(3-Amino-phenyl)-pyrimidin-2-yl]-[4-(2-methoxy-ethoxy)-phenyl]-amine [3];
[4-(3-Amino-phenyl)-pyrimidin-2-yl]-(4-nitro-phenyl)-amine [4];
(3-Nitro-phenyl)-[4-(3-nitro-phenyl)-pyrimidin-2-yl]-amine [5];
(4-Fluoro-phenyl)-[4-(3-nitro-phenyl)-pyrimidin-2-yl]-amine [6];
[4-(3-Amino-phenyl)-pyrimidin-2-yl]-(4-fluoro-phenyl)-amine [7];
N-[4-(3-Amino-phenyl)-pyrimidin-2-yl]-benzene-1,3-diamine [8];
N,N-Dimethyl-N′-[4-(3-nitro-phenyl)-pyrimidin-2-yl]-benzene-1,4-diamine [9];
N-Ethyl-N-{3-[2-(4-hydroxy-phenylamino)-pyrimidin-4-yl]-phenyl}-acetamide [10];
N-13-[2-(4-Hydroxy-phenylamino)-pyrimidin-4-yl]-phenyl-acetamide [11];
N-{3-[2-(4-Hydroxy-phenylamino)-pyrimidin-4-yl]-phenyl}-N-methyl-acetamide [12];
N-{3-[2-(4-Hydroxy-phenylamino)-pyrimidin-4-yl]-phenyl}-N-isobutyl-acetamide [13];
4-[4-(3-Methylamino-phenyl)-pyrimidin-2-ylamino]-phenol [14];
4-[4-(3-Amino-phenyl)-pyrimidin-2-ylamino]-phenol [15];
(4-Chloro-phenyl)-[4-(3-chloro-phenyl)-pyrimidin-2-yl]-amine [16];
4-[4-(3-Chloro-phenyl)-pyrimidin-2-ylamino]-phenol [17];
3-[4-(3-Chloro-phenyl)-pyrimidin-2-ylamino]-phenol [18];
[4-(3-Amino-phenyl)-pyrimidin-2-yl]-(3-nitro-phenyl)-amine [19];
N-[4-(3,4-Dichloro-phenyl)-pyrimidin-2-yl]-N′,N′-dimethyl-benzene-1,4-diamine [20];
4-[4-(3,4-Dichloro-phenyl)-pyrimidin-2-ylamino]-phenol [21];
3-[4-(3,4-Dichloro-phenyl)-pyrimidin-2-ylamino]-phenol [22];
N-Ethyl-N-{3-[2-(4-methoxy-phenylamino)-pyrimidin-4-yl]-phenyl}-acetamide [23];
N-Ethyl-N-13-[2-(4-nitro-phenylamino)-pyrimidin-4-yl]-phenyl-acetamide [24];
[4-(3-Ethylamino-phenyl)-pyrimidin-2-yl]-(4-methoxy-phenyl)-amine [25];
[4-(3-Ethylamino-phenyl)-pyrimidin-2-yl]-(4-nitro-phenyl)-amine [26];
{4-[3-(Benzylamino-methyl)-phenyl]-pyrimidin-2-yl}-(3-(3-nitro-phenyl)-amine [27];
3-{4-[3-(Benzylamino-methyl)-phenyl]-pyrimidin-2-ylamino}-phenol [28];
[4-(3-imidazol-1-ylmethyl-phenyl)-pyrimidin-2-yl]-(3-nitro-phenyl)-amine [29];
(3-Nitro-phenyl)-[4-(3-[1,2,4]triazol-1-ylmethyl-phenyl)-pyrimidin-2-yl]-amine [30];
[4-(3,4-Dichloro-phenyl)-pyrimidin-2-yl]-(3-nitro-phenyl)-amine [31];
(4-Morpholin-4-yl-phenyl)-[4-(3-[1,2,4]triazol-1-ylmethyl-phenyl)-pyrimidin-2-yl]-amine [32];
4-[4-(3-[1,2,4]Triazol-1-ylmethyl-phenyl)-pyrimidin-2-ylamino]-phenol [33];
3-[4-(3-[1,2,4]Triazol-1-ylmethyl-phenyl)-pyrimidin-2-ylamino]-phenol [34];
(3-Methoxy-phenyl)-[4-(3-[1,2,4]triazol-1-ylmethyl-phenyl)-pyrimidin-2-yl]-amine [35];
3-[4-(3-[1,2,4]Triazol-1-ylmethyl-phenyl)-pyrimidin-2-ylamino]-benzonitrile [36];
Phenyl-(4-phenyl-pyrimidin-2-yl)-amine [37];
[4-(5-Fluoro-2-methoxy-phenyl)-pyrimidin-2-yl]-phenyl-amine [38];
[4-(3-Morpholin-4-ylmethyl-phenyl)-pyrimidin-2-yl]-(3-nitro-phenyl)-amine [39];
N-{3-[2-(3-Nitro-phenylamino)-pyrimidin-4-yl]-benzyl}-methanesulfonamide [40];
(4-Nitro-phenyl)-[4-(3-[1,2,4]triazol-1-ylmethyl-phenyl)-pyrimidin-2-yl]-amine [41];
(4-Methoxy-phenyl)-[4-(3-[1,2,4]triazol-1-ylmethyl-phenyl)-pyrimidin-2-yl]-amine [42];
N,N-Dimethyl-N′-[4-(3-[1,2,4]triazol-1-ylmethyl-phenyl)-pyrimidin-2-yl]-benzene-1,4-diamine [43];
[4-(2,5-Dimethoxy-phenyl)-pyrimidin-2-yl]-(3-nitro-phenyl)-amine [44];
4-[4-(2,5-Dimethoxy-phenyl)-pyrimidin-2-ylamino]-phenol [45];
(4-{3-[(Ethyl-isopropyl-amino)methyl]-phenyl}-pyrimidin-2-yl)-(3-nitro-phenyl)-amine [46];
[4-(4-Chloro-3-[1,2,4]triazol-1-ylmethyl-phenyl)-pyrimidin-2-yl]-(3-nitro-phenyl)-amine [47];
{4-[3-(Benzylamino-methyl)-phenyl]-pyrimidin-2-yl}-(6-chloro-pyridin-3-yl)-amine [48];
[4-(3,4-Dichloro-phenyl)-pyrimidin-2-yl]-(6-methoxy-pyridin-3-yl)-amine [49];
(6-Methoxy-pyridin-3-yl)-[4-(3-[1,2,4]triazol-1-ylmethyl-phenyl)-pyrimidin-2-yl]-amine [50];
3-[2-(6-Methoxy-pyridin-3-ylamino)-pyrimidin-4-yl]-benzonitrile [51];
[4-(2,5-Dimethoxy-phenyl)-pyrimidin-2-yl]-(6-methoxy-pyridin-3-yl)-amine [52];
(4-{3-[(Ethyl-isopropyl-amino)-methyl]-phenyl}-pyrimidin-2-yl)-(6-methoxy-pyridin-3-yl)-amine [53];
{4-[3-(4-Methyl-piperazin-1-ylmethyl)-phenyl]-pyrimidin-2-yl}-(3-nitro-phenyl)-amine [54];
3-[2-(3-Nitro-phenylamino)-pyrimidin-4-yl]-phenol [55];
[3-[2-(3-Hydroxy-phenylamino)-pyrimidin-4-yl]-phenol [56];
3-[2-(3-Fluoro-phenylamino)-pyrimidin-4-yl]-phenol [57];
(6-Methoxy-pyridin-3-yl)-{4-[3-(4-methyl-piperazin-1-ylmethyl)-phenyl]-pyrimidin-2-yl}-amine [58];
[4-(3-Imidazol-1-ylmethyl-phenyl)-pyrimidin-2-yl]-(6-methoxy-pyridin-3-yl)-amine [59];
N-{3-[2-(3-Hydroxymethyl-phenylamino)-pyrimidin-4-yl]-phenyl}-acetamide [60];
[4-(2,5-Dimethyl-phenyl)-pyrimidin-2-yl]-(3-nitro-phenyl)-amine [61];
3-[4-(2,5-Dimethyl-phenyl)-pyrimidin-2-ylamino]-phenol [62];
[4-(2,5-Dimethyl-phenyl)-pyrimidin-2-yl]-(3-fluoro-phenyl)-amine [63];
3-[4-(3-Nitro-phenyl)-pyrimidin-2-ylamino]-phenol [64];
(3-Fluoro-phenyl)-[4-(3-nitro-phenyl)-pyrimidin-2-yl]-amine [65];
N-[3-(2-Phenylamino-pyrimidin-4-yl)-phenyl]-acetamide [66];
N-{3-[2-(3-Hydroxy-phenylamino)-pyrimidin-4-yl]-phenyl}-acetamide [67];
N-{3-[2-(3,5-Dimethoxy-phenylamino)-pyrimidin-4-yl]-phenyl}-acetamide [68];
N-{3-[2-(3-Nitro-phenylamino)-pyrimidin-4-yl]-phenyl}-acetamide [69];
N-{3-[2-(Pyridin-3-ylamino)-pyrimidin-4-yl]-phenyl}-acetamide [70];
[4-(3-Dimethylaminomethyl-phenyl)-pyrimidin-2-yl]-(3-nitro-phenyl)-amine [71];
3-[2-(3-Hydroxymethyl-phenylamino)-pyrimidin-4-yl]-phenol [72];
3-[2-(Pyridin-3-ylamino)-pyrimidin-4-yl]-phenol [73];
3-[2-(6-Methoxy-pyridin-3-ylamino)-pyrimidin-4-yl]-phenol [74];
3-[2-(3,5-Bis-trifluoromethyl-phenylamino)-pyrimidin-4-yl]-phenol [75];
3-[4-(4-Methoxy-phenyl)-pyrimidin-2-ylamino]-phenol [76];
[4-(3-Methoxy-phenyl)-pyrimidin-2-yl]-(6-methoxy-pyridin-3-yl)-amine [77];
N-Isopropyl-N-3-[2-(3-nitro-phenylamino)-pyrimidin-4-yl]-benzyl}-acetamide [78];
(1-{3-[2-(3-Nitro-phenylamino)-pyrimidin-4-yl]-benzyl}-piperidin-2-yl)-methanol [79];
3-[4-(3-Dimethylaminomethyl-phenyl)-pyrimidin-2-ylamino]-phenol [80];
4-[4-(3-Dimethylaminomethyl-phenyl)-pyrimidin-2-ylamino]-phenol [81];
[4-(3-Dimethylaminomethyl-phenyl)-pyrimidin-2-yl]-(4-morpholin-4-yl-phenyl)-amine [82];
[4-(3-Dimethylaminomethyl-phenyl)-pyrimidin-2-yl]-(6-methoxy-pyridin-3-yl)-amine [83];
[4-(3-Diethylaminomethyl-phenyl)-pyrimidin-2-yl]-(3-nitro-phenyl)-amine [84];
N-Methyl-3-nitro-N-3-[2-(3-nitro-phenylamino)-pyrimidin-4-yl]-benzyl}benzenesulfonamide [85];
(3-Nitro-phenyl)-{4-[3-(2-phenylaminomethyl-pyrrolidin-1-ylmethyl)-phenyl]-pyrimidin-2-yl}-amine [86];
[4-(3-Methoxy-phenyl)-pyrimidin-2-yl]-(3-nitro-phenyl)-amine [87];
3-[4-(3-Methoxy-phenyl)-pyrimidin-2-ylamino]-phenol [88];
4-[4-(3,4-Dimethoxy-phenyl)-pyrimidin-2-ylamino]-phenol [89];
[4-(3,4-Dimethoxy-phenyl)-pyrimidin-2-yl]-(3-nitro-phenyl)-amine [90];
{3-[2-(3-Nitro-phenylamino)-pyrimidin-4-yl]-phenyl}-methanol [91];
3-[2-(3-Hydroxy-phenylamino)-pyrimidin-4-yl]-benzonitrile [92];
3-[2-(4-Hydroxy-phenylamino)-pyrimidin-4-yl]-benzonitrile [93];
[4-(4-Methoxy-phenyl)-pyrimidin-2-yl]-(3-nitro-phenyl)-amine [94];
3-[4-(3-Trifluoromethyl-phenyl)-pyrimidin-2-ylamino]-phenol [95];
4-[4-(3-Trifluoromethyl-phenyl)-pyrimidin-2-ylamino]-phenol [96];
(3-Nitro-phenyl)-[4-(3-trifluoromethyl-phenyl)-pyrimidin-2-yl]-amine [97];
4-[4-(3-Methoxy-phenyl)-pyrimidin-2-ylamino]-phenol [98];
1-{3-[2-(3-Nitro-phenylamino)-pyrimidin-4-yl]-benzyl}-piperidine-3-carboxylic acid amide [99];
2-(1-{3-[2-(3-Nitro-phenylamino)-pyrimidin-4-yl]-benzyl}-piperidin-3-yl)-ethanol [100];
(1-{3-[2-(4-Morpholin-4-yl-phenylamino)-pyrimidin-4-yl]-benzyl}-piperidin-2-61)-methanol [101];
(1-{3-[2-(6-Methoxy-pyridin-3-ylamino)-pyrimidin-4-yl]-benzyl}-piperidin-2-yl)-methanol [102];
3-{4-[3-(2-Hydroxymethyl-piperidin-1-ylmethyl)-phenyl]-pyrimidin-2-ylamino}-phenol [103];
(3-Methanesulfonyl-phenyl)-[4-(3-[1,2,4]triazol-1-ylmethyl-phenyl)-pyrimidin-2-yl]-amine [104];
(1-{3-[2-(3-Nitro-phenylamino)-pyrimidin-4-yl]-benzyl}-piperidin-3-yl)-methanol [105];
4-{4-[3-(2-Hydroxymethyol-piperidin-1-ylmethyl)-phenyl]-pyrimidin-2-ylaminol}-phenol [106];
(1-{3-[2-(3,5-Bis-hydroxymethyl-phenylamino)-pyrimidin-4-yl]-benzyl}-piperidin-2-yl)-methanol [107];
(1-3-[2-(4-Methyl-3-nitro-phenylamino)-pyrimidin-4-yl]-benzyl}-piperidin-2-yl)-methanol [108];
3-[4-(4-Ethoxy-phenyl)-pyrimidin-2-ylamino]-phenol [109];
4-[4-(4-Methoxy-phenyl)-pyrimidin-2-ylamino]-phenol [110];
[4-(4-Methoxy-phenyl)-pyrimidin-2-yl]-(4-morpholin-4-yl-phenyl)-amine [111];
[4-(3-Chloro-phenyl)-pyrimidin-2-yl]-(3-nitro-phenyl)-amine [112];
4-[4-(3-Fluoro-phenyl)-pyrimidin-2-ylamino]-phenol [113];
3-[4-(2,5-Difluoro-phenyl)-pyrimidin-2-ylamino]-phenol [114];
3-[4-(3-Hydroxymethyl-phenyl)-pyrimidin-2-ylamino]-phenol [115];
{3-[2-(3-Fluoro-phenylamino)-pyrimidin-4-yl]-phenyl}-methanol [116];
{3-[2-(3,5-Dinitro-phenylamino)-pyrimidin-4-yl]-phenyl}-methanol [117];
(3-Fluoro-phenyl)-[4-(3-methoxy-phenyl)-pyrimidin-2-yl]-amine [118];
(3-Fluoro-phenyl)-[4-(4-methoxy-phenyl)-pyrimidin-2-yl]-amine [119];
3-[2-(3,5-Dimethoxy-phenylamino)-pyrimidin-4-yl]-phenol [121];
3-[2-(4-Hydroxy-phenylamino)-pyrimidin-4-yl]-phenol [122];
[4-(2,5-Difluoro-phenyl)-pyrimidin-2-yl]-(3-nitro-phenyl)-amine [123];
[4-(4-Methoxy-phenyl)-pyrimidin-2-yl]-(6-methoxy-pyridin-3-yl)-amine [124];
{3-[2-(6-Methoxy-pyridin-3-ylamino)-pyrimidin-4-yl]-phenyl}-methanol [125];
(3-Nitro-phenyl)-{4-[4-(2-[1,2,4]triazol-1-yl-ethyl)-phenyl]-pyrimidin-2-yl}-amine [126];
(1-4-[2-(3-Nitro-phenylamino)-pyrimidin-4-yl]-benzyl}-piperidin-2-yl)-methanol [127];
N-Methyl-N-{3-[2-(3-nitro-phenylamino)-pyrimidin-4-yl]-phenyl}-methanesulfonamide [129];
N-{3-[2-(3-Hydroxy-phenylamino)-pyrimidin-4-yl]-phenyl}-N-methylmethanesulfonamide [130];
N-{3-[2-(4-Hydroxy-phenylamino)-pyrimidin-4-yl]-phenyl}-N-methyl-methanesulfonamide [131]; and
N-{3-[2-(6-Methoxy-pyridin-3-ylamino)-pyrimidin-4-yl]-phenyl}-N-methyl-methanesulfonamide [132].

5. The method of claim 1, wherein said pain comprises chronic pain, inflammatory pain, neuropathic pain, or a combination thereof.

6. A pharmaceutical composition comprising at least one inhibitor of a cyclin-dependent kinase for the treatment of any type of pain, wherein said at least one inhibitor is characterized by general Formula I, 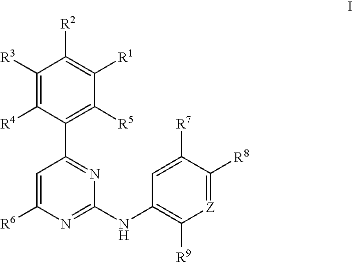
wherein:
Z is CR¹⁰ or N;
one of R¹ and R² is selected from (CH₂)ₘR¹¹, (CH₂)ₘR¹², (CH₂)ₘNR¹²R¹³, (CH₂)ₘOR¹², (CH₂)ₘNR¹³CO(CH₂)ₙR¹¹, (CH₂)ₘNR¹³COR¹², (CH₂)ₘCONR¹³(CH₂)nR¹¹, (CH₂)ₘCONR¹²R¹³, (CH₂)ₘCO(CH₂)ₙR¹¹ and (CH₂)COR¹²; where m is 0, 1, 2, 3 or 4, and n is 1, 2, 3 or 4;
the other of R¹ and R² is H or R¹¹;
R³ and R⁵ are both H;
R⁴ is H or R¹¹;
R⁶ is H or (CH₂)ₚR¹¹, where p is 0 or 1;
R⁷, R⁹ and R¹⁰ are each independently H or R¹¹;
R⁸ is selected from H, halogen, NO₂, CN, OR¹³, NR¹³R¹⁴, NHCOR¹³, CF₃, COR¹³, R¹³, CONR¹³R¹⁵, SO₂NR¹³R¹⁴, SO₂R¹³, NR¹³SO₂R¹⁴, OCH₂CH₂OH, OCH₂CH₂OMe, morpholine, piperidine, and piperazine;
each R¹¹ is independently halogen, NO₂, CN, (CH₂)_{q}OR¹³, (CH₂)ᵣNR¹³R¹⁴, NHCOR¹³, CF₃, COR¹⁴, R¹³, CONR¹³R¹⁴, SO₂NR¹³R¹⁴, SO₂R¹³, OR¹², NR¹³SO₂R¹⁴, OCH₂CH₂OH, OCH₂CH₂OMe, NR¹³SO₂R¹³, (CH₂)ₛNR¹²R¹³, morpholine, piperidine or piperazine, where q, r and s are each independently 0, 1, 2, 3 or 4;
each R¹² is independently a hydrocarbyl group optionally containing one or more heteroatoms and optionally substituted with one or more R¹¹ groups;
each R¹³ and each R¹⁴ is independently H or an alkyl group; and
R¹⁵ is an alkyl group;
provided that:
when -Z is CR¹⁰ and R⁹ is H, then at least one of R⁷, R⁸ and R¹⁰ is other than OMe; and
when -Z is CR¹⁰ and R⁷, R⁸, and R⁹ are all H, then R¹⁰ is other than OCF₂CHF₂;
and the N-oxide derivatives, prodrug derivatives, protected derivatives, individual isomers, mixtures of isomers, enantiomers, diastereomers thereof, and the pharmaceutically acceptable salts and solvates of such compounds.

7. The pharmaceutical composition of claim 6, wherein said pain comprises chronic pain, inflammatory pain, neuropathic pain, or a combination thereof.
